(19) United States
(12) Patent Application Publication
(10) Pub. No.: US 2015/0216705 A1
(43) Pub. Date: Aug. 6, 2015

(54) UPPER TORSO PROTECTIVE ORTHOTIC DEVICES AND USES THEREOF

(71) Applicant: Paul EVANS, Mount Waverley, Victoria (AU)

(72) Inventor: Paul Evans, Mount Waverley (AU)

(21) Appl. No.: 14/421,539

(22) PCT Filed: Aug. 15, 2013
(86) PCT No.: PCT/AU2013/000899

§ 371 Date: Feb. 13, 2015

(30) Foreign Application Priority Data
Aug. 15, 2012 (AU) 2012903508
Feb. 22, 2013 (AU) 2013900607
Apr. 26, 2013 (AU) 2013901467

(51) Int. Cl. A61F 5/02

(52) U.S. Cl. CPC A61F 5/02

(57) ABSTRACT

Disclosed herein is a protective device which comprises an orthosis configured to be received against the upper front torso of a subject adjacent the subject's breasts, so as to restrict or eliminate stress on the breasts resulting from an external load applied to the upper front torso, the device including means for removably holding the orthosis to the subject or to an object applying the load such that the orthosis is so received. Also disclosed herein is an orthosis arranged to be received between the upper front torso of a subject and a support through which a load is applied to the upper front torso, to restrict or eliminate stress on the subject's breasts resulting from the load, the orthosis comprising a generally hollow resiliently deformable body defined by a wall of the orthosis, the body being arranged to contact the subject adjacent the breasts, and resiliently deformable ribbing within the body to support the wall, whereby the orthosis deforms resiliently when so received.

### FIELD OF THE INVENTION

The present invention relates to improved means for protecting the upper torso, including in particular the breasts, tissue adjacent the breasts and/or organs within the upper torso.

Embodiments of the invention provide orthoses configured to distribute, through a person's thoracic cage, compressive loads applied to the person's upper torso.

The invention has particular, though by no means exclusive, application to people suffering from mastalgia (breast pain) and people who have had mastectomies or breast augmentation or reconstruction surgery.

Preferred embodiments of the invention provide orthoses which are particularly useful, inter alia, in a clinical setting, such as during clinical examination and/or treatment, during radiological or other procedures, or when undertaking physical therapy or massage. Preferred embodiments of the invention also provide orthoses which have application for personal use outside the clinic such as, without limitation, during mastalgia.

Reference to any prior art in this specification is not, and should not be taken as, an acknowledgment or any form of suggestion that this prior art forms part of the common general knowledge in any country.

### BACKGROUND

Breasts are composed of glandular, fatty, and fibrous tissue positioned over the pectoral muscles of the chest wall to which they are attached by fibrous Cooper's ligaments. Fatty tissue surrounds the breast glands extending throughout the breast, giving it a soft consistency.

Face-downward resting such as when prone, either in a clinical setting such as during physical therapy or massage, or simply relaxing such as when sunbathing can produce pain and discomfort to the breast tissue through compression and displacement of breast tissue that is sensitive to pain. This problem is exacerbated at times when the breasts are particularly tender or when the anatomical structure has been altered.

Breast tenderness (such as resulting from mastalgia, mastodynia or mammalgia) can increase, for example, during menstruation, pregnancy, puberty or breastfeeding, in the presence of mastitis or fibrocystic breast tissue, or as an adverse reaction to medications.

Patients post-mastectomy with extensive scar tissue experience pain when prone. Patients with breast augmentation report that compression and displacement of breast, scar and implant tissue produces pain when prone. Indeed, such compression or displacement of breast tissue can cause complications such as damage to the implant and/or movement of the implant and damage to adjacent tissue.

Correct breast positioning is crucial, for example, to decrease discomfort during mammography or other radiological investigation, and also improves the accuracy of the investigation. Further, chest wall imaging is enhanced if the breast is maintained in a proper position during radiological investigation.

Treatment tables and other devices have previously been produced to address some of these problems by providing direct cushioning or generally breast-shaped recesses for breasts. Prior art devices have generally failed to allow successfully for variations in breast position and size and skeletal variations amongst the population.

The breasts can also be subjected or vulnerable to undesirable compression or displacement when the person is not resting prone on a support surface, e.g. when the person is seated in transit and is restrained by a seatbelt or other means of restraint received against the upper anterior torso, or when the person is engaged in physical activity, e.g. playing contact sports.

### SUMMARY OF THE INVENTION

According to a first aspect of the present invention, there is provided a protective device which comprises an orthosis configured to be received against the upper front torso of a subject adjacent the subject's breasts, so as to restrict or eliminate stress on the breasts resulting from an external load applied to the upper front torso, the device including means for removably holding the orthosis to the subject or to an object applying the external load such that the orthosis is so received.

The external load will generally comprise a compressive load.

Stresses on the breasts which are restricted or eliminated by preferred embodiments of the invention may include, for example, compressive stresses, and also shear stresses, torsional stresses or prying stresses, particularly within the breasts. Such stresses can be particularly high and/or of particular concern where the breasts are provided with implants (which can, among other things, twist and warp when the breasts are loaded).

The device may be configured such that the receipt of the orthosis against the upper front torso is either direct or indirect, the orthosis in the latter case not lying in direct contact with the subject.

Preferably, the orthosis comprises a generally hollow resiliently deformable body defined by a wall of the orthosis, the body being arranged to contact the subject adjacent the breasts, and resiliently deformable ribbing within the body to support the wall, whereby the orthosis deforms resiliently under the external load.

Alternatively, the orthosis may comprise any one of the orthoses described in international patent application no. PCT/AU2011/000233 (publication no. WO2011/106836A1) and illustrated in FIGS. 1, 2, 5A 5B and 6 of that application. The content of that application is incorporated herein by reference in its entirety.

In one embodiment of the invention, said means comprises means for removably holding the orthosis to the subject.

In one embodiment of the invention, the device is receivable around the subject's neck such that the orthosis is hung from the neck. In such an embodiment, the device preferably includes a lanyard receivable around the neck and supporting the orthosis such that it hangs from the neck.

In one embodiment of the invention, the means for removably holding the orthosis to the subject comprises means for attaching the orthosis to clothing worn by the subject over the front upper torso. Said means for attaching may comprise, for example, one or more fasteners. The or each fastener may comprise, for example, a clip or a hook fastener engageable with a respective loop fastener provided on or defined by the clothing or vice versa.

In another embodiment of the invention, the device comprises a harness which holds the orthosis and is wearable over the torso.

In one preferred embodiment of the invention, the device comprises a vest which holds the orthosis. The orthosis may be removably fitted to the vest, e.g. via at least one holder releasably holding it to the vest. The or each holder may comprise a pocket, strap or loop received around a portion of the orthosis. In an alternative preferred embodiment of the invention, the orthosis may be a permanent fixture in the vest; for example, it may be moulded into, and/or integrally formed with, the vest.

In a preferred embodiment of the invention, the device includes strapping for holding it to the subject. Preferably, the strapping comprises opposite shoulder straps.

The device may include a front panel against an inner side of which the orthosis is positioned, the panel being arranged to impart the compressive load to the orthosis. The panel may be configured to distribute the load through the orthosis; to this end, the panel may be rigid or semi-rigid. Preferably, however, the panel is flexible. The panel may be resiliently flexible. Preferably, the or each holder is arranged to hold the orthosis against the panel.

Preferably, the device is configured so as not to entrap the subject's breasts circumferentially.

In one embodiment of the invention, said means comprises means for removably holding the orthosis or to an object applying the external load. Said means preferably comprises a mounting configured to couple the orthosis to the object.

According to a second aspect of the present invention, there is provided said protective device attached to the subject or object such that the orthosis is received against the upper front torso adjacent the breasts so as to restrict or eliminate stress on the breasts resulting from said compressive load.

In one embodiment of the invention, the subject is in motion, and applied over subject's upper front torso is a support restraining the person against harmful movement resulting from a sudden stop or change in said motion, the external compressive load being applied by the support. For example, the subject may be seated, such as in a vehicle, and the support may comprise a seatbelt. The means for removably holding the attachment may hold the orthosis to the subject or, alternatively, may hold the attachment to the seatbelt. The seatbelt may comprise, for example, a sash-type seatbelt or a 3-, 4-, 5-, 6- or 7-point seatbelt. The device may decrease the severity and incidence of Seatbelt Syndrome.

In another embodiment of the invention, the subject is engaged in physical activity which exposes the upper front torso to compressive loads or to a possibility of such loads. The physical activity may comprise, for example, sport, such as contact sport.

The device can be used in any one of various applications in which loading is required to be redistributed away from the pressure-sensitive and mechanically intolerant or weakened breast tissue and/or implant material.

According to a third aspect of the present invention, there is provided an orthosis arranged to be received between the upper front torso of a subject and a support through which a load is applied to the upper front torso, to restrict or eliminate stress on the subject's breasts resulting from the load, the orthosis comprising a generally hollow resiliently deformable body defined by a wall of the orthosis, the body being arranged to contact the subject adjacent the breasts, and resiliently deformable ribbing within the body to support the wall, whereby the orthosis deforms resiliently when so received.

According to a fourth aspect of the present invention, there is provided a method of restricting or eliminating stress on the breasts of a subject resulting from an external load applied to the upper front torso of the subject, wherein an orthosis as defined above is held against the subject adjacent the breasts to transfer force from the load to the subject's thoracic cage. The load may be exerted by a support and the orthosis arranged between the support and the thoracic cage.

The orthosis may be one which comprises said body and ribbing, and either the body or a base of the orthosis arranged so as to contact the subject. Alternatively, the orthosis may comprise any one of the abovementioned orthoses disclosed in international patent application no. PCT/AU2011/000233.

According to a fifth aspect of the present invention, there is provided a method of restricting or eliminating stress on the breasts of a subject resulting from loading on the subject's upper front torso exerted by a support, wherein an orthosis comprising said body and ribbing as defined above is arranged between the upper front torso and the support whereby the body contacts the subject adjacent the breasts such that the orthosis deforms resiliently.

Preferably, said base is substantially flat.

The support may be defined by a surface, against which the subject lies prone, such as, for example, a clinical table. Alternatively, the support may be defined by a restraint securing the subject against harmful movement resulting from a sudden stop or change in said motion, the external compressive load being applied by the restraint; for example, the subject may be seated, such as in a vehicle, and the support may comprise a seatbelt. The seatbelt may comprise, for example, a sash-type seatbelt or a 3-, 4-, 5-, 6- or 7-point seatbelt. The orthosis may decrease the severity and incidence of Seatbelt Syndrome.

The subject may lie prone so as to rest against the support. Alternatively, the orthosis may be used when the subject is in other than a prone position, and mechanical loading by the support (which may, for example, exert the loading through a generally upright surface thereof) is required to be redistributed away from the pressure-sensitive and mechanically intolerant breast and/or implant material.

In any of the third, fourth and fifth aspects, the orthosis may be that, or other than that, of said protective device.

The orthosis according to the preferred embodiments of the invention comprises a said wall having a hardness which is such that it provides “soft-feel” characteristics.

Preferably, the ribbing comprises ribs which extend to a base of the orthosis to be compressed between the wall/body and the support or a surface thereof. Preferably, the base is generally open. Preferably, the base is substantially flat. Preferably, the ribs extending to the base comprises ones which are anchored, either directly or indirectly, to the body/wall substantially around. entireties of peripheries thereof within the body.

In the preferred embodiments of the invention, the orthosis is formed to engage the subject in a cushioning manner.

Preferably, the ribbing is integrally formed with the wall.

Preferably, the orthosis is formed by injection moulding. Preferably, the orthosis is moulded as a single piece.

In the preferred embodiments of the present invention, the orthosis is formed from thermoplastic elastomeric material, which is preferably medical grade and may comprise, for example, a thermoplastic elastomer marketed under the name Mediprene or Thermolast-M. Preferably, at least said body is formed from medical grade thermoplastic elastomer. More generally, however, the orthosis may be formed from any one or more of several resiliently deformable materials, including, without limitation, a natural or synthetic elastomeric material, e.g. a rubber modified polypropylene such as a mineral filled, UV stabilised thermoplastic styrenic rubber modified polypropylene compound that has been oil extended. Suitable compositions are known to those of skill in the field of polymeric materials. These include those formed from natural latex, isoprenes, polyisoprenes, polystyrene butadienes, nitrile elastomers, butyl elastomers, ethylene propylene terpolymers, silicone elastomers, neoprenes, polysulfide, poly acetyl, eperchloride, fluoroelastomers, hypalon, halogenated butyl, polyurethanes, and thermoplastic elastomers. Polyurethane elastomers (spandex) are made from urethane (for strength) and polyether or polyester (for elasticity). They are versatile elastomers because of their hardness, strength and oil resistance. They are widely used for making foam for bedding. Nitrile elastomers are co-polymers of acrylonitrile and a diene, such as butadiene. Neoprene (polychloroprene) is useful for heavy-duty applications. Butyl elastomers are co-polymers of isobutylene and isoprene (1.3%). These are typically used to make injection models and extruded products where good shock absorption is required. They are also resistant to abrasion. Styrene-butadiene co-polymers (BunaS and SBR) are formed by emulsion polymerisation of butadiene and styrene in various ratios, cis-polybutadiene and cis-polyisoprene. Polymers are also usefully employed. Ethylene-propylene co-polymers are similar to SBR. They are often used for car parts and footwear. Silicone elastomers are polymers of dimethysilanediol. Polysulfide, sulfide and polyacrylate elastomers have valuable characteristics. These are used in inter alia medical implants.

Preferably, the orthosis comprises:
(a) a central portion extending along a cephalad-caudal axis of the orthosis to be received between the breasts to support the sternum; and
(b) opposed portions extending bilaterally outwardly from a cephalad end of the central portion to support the clavicle and/or upper ribcage/costal cartilage (“cephalad portions”); and/or
(c) opposed portions extending bilaterally outwardly from a caudal end of the central portion to support the lower ribcage/costal cartilage and/or upper abdomen (“caudal portions”),
each portion comprising a respective portion of the body and part of the ribbing and being configured such that spaces are defined laterally outward of the cephalad-caudal axis and the breasts occupy the spaces.

Preferably each said part of the ribbing comprises one or more ribs extending to a base of the orthosis to be compressed between the wall/body and a surface of an object, such as a support, through which the compressive load to the upper torso is imparted.

The orthosis may then, advantageously, be configured so as to engage the subject in a manner such that, regardless of the subject's size, there is no circumferential entrapment of the subject's breasts. The orthosis may be configured such that the breasts, to some extent, are displaced laterally and/or superolaterally to occupy the spaces, particularly where the subject is lying prone.

Preferably, the central portion ribbing comprises one or more transverse ribs (“central portion transverse ribs”) extending transverse to the cephalad-caudal axis. Preferably, the central portion ribbing comprises a plurality of transverse ribs spaced apart along said cephalad-caudal axis. Preferably, the number of central portion transverse ribs is three.

Preferably, the central portion transverse ribs comprise ones which are bowed in a direction along the cephalad-caudal axis. Preferably, the bowed central portion transverse ribs are bowed symmetrically about that axis. Preferably, each bowed central portion transverse rib is bowed to an extent generally commensurate with a distance that that rib is spaced froth an axis which is perpendicular to the cephalad-caudal axis and extends through a narrowest section of the central portion (“the perpendicular axis”). Preferably, each bowed central portion transverse rib is bowed in the same direction as that in which it is spaced from the perpendicular axis. Preferably, the central portion transverse ribs comprise ones which are arranged both caphalad and caudal of the perpendicular axis.

Preferably, each central portion transverse rib extends substantially the depth of the body interior where that rib is located. Preferably, each of the central portion transverse ribs interconnects opposed longitudinal wall sections of the central portion. Preferably, each of the central portion transverse ribs is anchored around an entirety of a periphery thereof which is contained in the interior. Preferably, each central portion transverse rib extends to a base of the orthosis to be compressed between the wall/body and a surface of an object, such as a support, through which the compressive load to the upper front torso is imparted.

Preferably, the central portion ribbing further comprises at least one longitudinal rib (“central portion longitudinal rib”) interconnected with the central portion transverse ribs to stabilise them. The or each central portion longitudinal rib may extend parallel to or in alignment with the cephalad-caudal axis. The at least one longitudinal rib may consist of a single central portion longitudinal rib which is centred over or coaxial with the cephalad-caudal axis, or may instead comprise a pair of ribs arranged, preferably symmetrically, to either side of that axis, each rib of said pair preferably being bowed laterally inwardly. Preferably, the or each central portion longitudinal rib is attached to the wall and projects downwardly therefrom. Preferably, the or each central portion longitudinal rib extends only partway through, e.g. about a third to about a half of, the depth of the body interior where that rib is located.

Preferably, the central portion transverse and longitudinal ribs define a lattice structure.

The cephalad-caudal axis may define a central and/or longitudinal axis of the orthosis.

Preferably, the central portion is outwardly convex along its length, i.e. as viewed in section orthogonal to the longitudinal axis.

Preferably, the orthosis includes the caudal portions.

Preferably, the caudal portions are configured to contact the subject along axes which are divergent about the cephalad-caudal axis in a caudal direction. Preferably, each of the divergent axes forms an angle with the longitudinal axis of between about 65° and about 85°. More preferably, each angle is between about 70° and about 80°. More preferably still, each angle is about 75°.

Preferably, the ribbing of each caudal portion comprises one or more transverse ribs (“caudal portion transverse ribs”) arranged transverse to a respective one of the divergent axes along which the caudal portion is configured to contact the subject. Preferably, the ribbing of each caudal portion comprises a plurality of caudal portion transverse ribs spaced apart in a direction generally aligned with that respective axis. In one embodiment of the invention, the number of caudal portion transverse ribs which each caudal portion comprises is five. In another embodiment of the invention, that number is three.

Preferably, the transverse ribs of each caudal portion comprise ones which are bowed in a direction towards a distal end of the caudal portion. Preferably, each bowed transverse rib of each caudal portion is bowed to an extent generally commensurate with a distance that that rib is spaced from a proximal end of the caudal portion in a direction towards the distal end of the caudal portion. Preferably, each bowed caudal portion transverse rib is bowed in that direction.

Preferably, each caudal portion transverse rib extends substantially the depth of the body interior where that rib is located. Preferably, each of the transverse ribs of each caudal portion interconnects opposed longitudinal wall sections of that caudal portion. Preferably, each of the caudal portion transverse ribs is anchored around an entirety of a periphery thereof which is contained in the interior. Preferably, each caudal portion transverse rib extends to a base of the orthosis to be compressed between the wall/body and a surface of an object, such as a support, through which the compressive load to the upper front torso is imparted.

Preferably, the ribbing of each caudal portion further comprises at least one longitudinal rib (“caudal portion longitudinal rib”) interconnected with the transverse ribs of the caudal portion to stabilise them. Preferably, the or each longitudinal rib of each caudal portion extend in general alignment with the respective one of the divergent axes along which the caudal portion is configured to contact the subject. Preferably, the at least one longitudinal rib consists of a pair of transversely spaced apart longitudinal ribs. Preferably, the or each caudal portion longitudinal rib is attached to the wall and projects downwardly therefrom. Preferably, the or caudal portion longitudinal rib extends only partway through, e.g. about a third to about a half of, the depth of the body interior where that rib is located.

Preferably, the caudal portion transverse and longitudinal ribs define a lattice structure.

Preferably, the caudal portions are integrally formed with the central portion.

Preferably, each of the caudal portions is outwardly convex as viewed in section orthogonal to an axis along which it contacts the subject.

Preferably, adjacent edge portions of the central and caudal portions form curves which define laterally inner caudal boundaries of the spaces. The edge portions may thus have a curved profile as viewed in section parallel a surface of an object, such as a support, when the orthosis is in use.

In the preferred embodiments of the invention, a general centre of curvature of each curve is superolateral of a position at which the respective adjacent edge portions meet.

Preferably, the orthosis is configured with a relief at/adjacent proximal ends of the caudal portions and/or at/adjacent a caudal end of the central portion to limit or eliminate stress/pressure on the xyphoid process. Preferably, the relief comprises a concavity in an outer surface of the body.

Preferably, the orthosis includes the cephalad portions.

Preferably, the cephalad portions are configured to contact the subject along axes which are divergent about the cephalad-caudal axis in a cephalad direction. Preferably, each of these axes forms an angle with the cephalad-caudal axis of between about 50° and about 70°. More preferably, each angle is between about 55° and about 65°. More preferably still, each angle is about 60°.

Preferably, the ribbing of each cephalad portion comprises one or more transverse ribs (“cephalad portion transverse ribs”) arranged transverse to a respective one of the divergent axes along which the cephalad portion is configured to contact the subject. Preferably, the ribbing of each cephalad portion comprises a plurality of cephalad portion transverse ribs spaced apart in a direction generally aligned with that respective axis. In one embodiment of the invention, the number of transverse ribs which each cephalad portion comprises is three. In another embodiment of the invention, that number is two.

Preferably, the transverse ribs of each cephalad portion comprise ones which are bowed in a direction towards a distal end of the cephalad portion. Preferably, each bowed transverse rib of each cephalad portion is bowed to an extent generally commensurate with a distance that that rib is spaced from a proximal end of the cephalad portion in a direction towards the distal end of the cephalad portion. Preferably, each bowed cephalad portion transverse rib is bowed in that direction.

Preferably, each cephalad portion transverse rib extends substantially the depth of the body interior where that rib is located. Preferably, each of the transverse ribs of each cephalad portion interconnects opposed longitudinal wall sections of that cephalad portion.

Preferably, each of the cephalad portion transverse ribs is anchored around an entirety of a periphery thereof which is contained in the interior. Preferably, each cephalad portion transverse rib extends to a base of the orthosis to be compressed between the wall/body and a surface of an object, such as a support, through which the compressive load to the upper front torso is imparted.

Preferably, the ribbing of each cephalad portion further comprises at least one longitudinal rib (“cephalad portion longitudinal rib”) interconnected with the transverse ribs of the cephalad portion to stabilise them. Preferably, the or each longitudinal rib of each cephalad portion extend in general alignment with the respective one of the divergent axes along which the cephalad portion is configured to contact the subject. Preferably, the at least one longitudinal rib consists of a pair of transversely spaced apart longitudinal ribs. Preferably, the or each cephalad portion longitudinal rib is attached to the wall and projects downwardly therefrom. Preferably, the or cephalad portion longitudinal rib extends only partway through, e.g. about a third to about a half of, the depth of the body interior where that rib is located.

Preferably, the ribbing includes a continuous rib arranged adjacent to and radially inward of a peripheral edge of the body/wall and extending in general alignment therewith, an edge of the continuous rib being coplanar with said peripheral edge, whereby the orthosis includes a peripheral seating portion, comprising a peripheral edge portion of the body/wall and the continuous rib, which is configured to remain in contact with a surface of an object, such as a support, through which the compressive load is imparted to the upper front torso when the orthosis is loaded, owing to a degree of relative movement between the edges of the continuous rib and body being permitted. Preferably, the ribbing further includes radial ribs, arranged at spaced positions around the seating portion and interconnecting the peripheral edge portion and peripheral rib, the radial ribs forming part of the peripheral seating portion. Preferably, edges of the radial ribs are coplanar with said peripheral edge.

Preferably, the cephalad portion transverse and longitudinal ribs define a lattice structure.

Preferably, the cephalad portions are integrally formed with the central portion.

Preferably, each of the cephalad portions is outwardly convex as viewed in section orthogonal to an axis along which it contacts the subject.

Preferably, adjacent edge portions of the central and cephalad portions form curves which define laterally inner cephalad boundaries of the spaces. The edge portions may thus have a curved profile as viewed in section parallel to the supporting surface when the orthosis is in use.

In the preferred embodiments of the invention, a general centre of curvature of each curve is sublateral of a position at which the respective adjacent edge portions meet.

Preferably, the orthosis is configured with a relief at/adjacent proximal ends of the cephalad portions and/or at/adjacent a cephalad end of the central portion to limit or eliminate stress/pressure on the trachea and/or jugular notch to facilitate breathing.

In preferred embodiments of the invention, the orthosis is separately formed from the surface of an object, such as a support, through which the compressive load is imparted to the upper front torso and able to be placed thereagainst for use. Preferably, the orthosis in these embodiments comprises a substantially open and/or flat base receivable against the surface.

In an alternative embodiment of the present invention, the orthosis could be attached to e.g. integrally formed with, the object.

Preferably, the cephalad portions are outwardly convex.

Preferably, the orthosis is configured with a relief between the cephalad portions to limit or eliminate stress/pressure on the trachea and/or jugular notch to facilitate breathing.

Preferably, the cephalad portions are configured to contact the subject along axes which are divergent about the longitudinal axis in a cephalad direction. Preferably, each of these axes forms an angle with the longitudinal axis of between about 50° and about 70°. More preferably, each angle is between about 55° and about 65°. More preferably still, each angle is about 60°.

Preferably, the cephalad portions are outwardly convex as viewed in section orthogonal to the axes.

Preferably, the body opens bilaterally outwardly of the central portion whereby it supports the sternum, the clavicle and/or upper ribcage/costal cartilage, and the lower ribcage/costal cartilage and/or upper abdomen. Preferably, the body is of a generally I-shaped configuration. Owing to a resulting laterally outwardly open configuration of the orthosis, the orthosis can engage the subject in a manner such that whatever the subject's size and proportions, that there is no circumferential entrapment of the breasts.

Preferably, the orthosis is formed from resiliently deformable material.

Preferably, the body is configured with smoothly curved edge portions which define substantially C-shaped and reverse C-shaped laterally inner boundaries respectively of the spaces.

According to a further aspect of the present invention, there is provided an orthosis or orthotic device as defined above in use.

In some embodiments of the invention, the orthosis comprises, in addition to the central portion, the caudal portions but the cephalad portions are omitted. In other embodiments of the invention, the orthosis comprises, in addition to the central portion, the cephalad portions but the caudal portions are omitted. Preferably, however, the orthosis comprises the central portion, the caudal portions and the cephalad portions.

In some embodiments, the orthosis in use is positioned against the sternum and clavicle and/or upper ribcage/costal cartilage, or alternatively, or in addition, under the lower ribcage/costal cartilage and/or abdomen, and receives and distributes compression loads away from pain-sensitive breast tissue and onto less pain-sensitive parts of the body adjacent to the orthosis.

Preferably, the orthosis is unitary. Alternatively, detachable connectors between portions are possible. Suitable connectors may include, without limitation, a cooperative latching arrangement configured between portions (e.g. Velcro), or a male-female type attachment arrangement attached to or within the orthosis portions (e.g. snap connection), a magnetic fastener arrangement and the like. Detachable connectors may themselves be detachable and may be sold separately or together as part of the subject kits.

Preferably, the orthosis is symmetrical about the cephalad-caudal axis. Preferably, the orthosis is resiliently deformable. Preferably, the orthosis is of molded construction.

The above summary is not and should not be seen in any way as an exhaustive recitation of all embodiments of the present invention.

Preferred embodiments of the invention provide an orthosis which is configured to deform resiliently when engaged by the subject, and to cushion the subject when so engaged.

As known in the art, the sternum is an elongate flattened bone that forms the middle portion of the anterior wall of the thorax. Its anterior end supports the clavicle and its margins articulate with the costal cartilage of the first seven pairs of ribs. It consists of three parts: most superiorly the “manubrium” which has a quadrangular shape narrowing to meet a long thin “body”, and the most inferior “xyphoid process”.

The costal cartilages are bars of hyaline-rich cartilage that provide considerable elasticity to the walls of the thorax. The lateral side of each cartilage is continuous with the rib bone. In older subjects, there is a tendency for at least the surface of the costal cartilage to undergo ossification.

In some embodiments, the orthosis has surface portions defining concavities which receive the breasts and are shaped to conform with the breasts when the breasts are received thereby.

An orthosis according to one preferred embodiment of the invention is configured to lie on a support surface to be received adjacent the breast of the subject resting face down on the surface, and comprises (a) a central sternum portion that rests on the supporting surface and extends upwardly to contact the sternum of the subject and axially along the midline between breasts; and (b) an upper clavicle portion that rests on the supporting surface and extends upwardly to contact the clavicle and/or upper ribcage/costal cartilage cephalad of the breasts; and (c) a lower ribcage portion that rests on the supporting surface and extends upwardly to contact the lower ribcage/costal cartilage and/or abdomen caudal of the breasts.

In some embodiments, the portion (a) extends along at least the length of the body of the sternum. In some embodiments, the portion (a) extends along the length of the body of the sternum and under the length of manubrium. In some embodiments, portion (a) and/or (c) are configured so as not to contact the xyphoid process; e.g., the orthosis may terminate above the process or be tapered in the region of the xiphoid process. In some embodiments, portion (c) extends over the lower ribcage/costal cartilage. In some embodiments, portion (b) extends over the clavicle. In some embodiments, the depth of the orthosis is substantially uniform axially along the sternum portion. In some embodiments, an orthosis comprising is manufactured as a single unit.

In a particular embodiment, the orthosis is open sided to allow for lateral displacement of breast tissue, and preferably unimpeded lateral displacement towards the anterior or auxiliary folds.

According to a further aspect of the present invention, there is provided a set comprising orthoses each of which accords with the invention, the orthoses being different-sized for supporting subjects having different breast sizes. The number of orthoses is preferably three to accommodate A-G cup sizing. Thus, orthoses and portions are manufactured to suit subjects with bra cup sizes from A to G and above. In some embodiments, the orthosis is customised to fit a particular subject. The set may be or form part of a kit.

In a preferred embodiment of the invention, there is provided, in accordance with said second aspect, a said protective device or orthosis in use, wherein the orthosis has been selected from the set, according to the size of the subject's breasts.

In some embodiments, the orthosis is used during a radiological or other procedure involving the breasts, such as during mammography or chest imaging.

Given the open side arrangement of orthoses according to preferred embodiments of the invention, those orthoses will be suitable for a wide range of subject sizes.

According to a further aspect of the present invention, there is provided a method of manufacturing an orthosis as defined above, the method comprising forming the orthosis by moulding. Preferably, the moulding comprises injection moulding.

Preferably, in each aspect of the invention, the orthosis is configured to abut the subject's thoracic cage so as to transfer force from the load thereto.

Although, the invention, as will be clear from the above discussion, is particularly application to women, it also has application to men, including in particular men who have had breast surgery.

### BRIEF DESCRIPTION OF THE DRAWINGS

The present invention will now be described, by way of non-limiting example only, with reference to the accompanying drawings, in which:

FIG. 1 shows upper perspective views of different-sized orthoses according to preferred embodiments of the present invention;

FIG. 2 shows side elevation views of the orthoses illustrated in FIG. 1;

FIG. 3 is an upper perspective view of a medium-sized orthosis according to a first of the preferred embodiments of the invention;

FIG. 4 is a top plan view of the orthosis of the first embodiment;

FIG. 5 is a bottom plan view of the orthosis of the first embodiment;

FIG. 6 is a lower perspective view of the orthosis of the first embodiment;

FIG. 7A is a view through section X-X indicated in FIG. 3;

FIG. 7B is a view of detail A indicated in FIG. 7A;

FIG. 8 is a plan view of the orthosis of the first embodiment showing particular dimensions thereof;

FIG. 9 is a bottom plan view of a small-sized orthosis according to a second of the preferred embodiments of the invention;

FIG. 10 is a top plan view of the orthosis of the second embodiment showing particular dimensions thereof;

FIG. 11 is a bottom plan view of a large sized orthosis according to the third of the preferred embodiments of the invention;

FIG. 12 is a top plan view of the orthosis of the third embodiment showing particular dimensions thereof;

FIG. 13 is a bottom plan view showing an orthosis embodying the invention in use;

FIG. 14 is a perspective view showing an orthosis embodying the invention in use;

FIGS. 15A and 15B are comparative views showing the manner in which the orthosis in use prevents compression and undesirable displacement of the breasts;

FIGS. 16A and 16B illustrate the manner in which ribs of an orthosis embodying the invention deform when the orthosis is in use;

FIG. 17 is a front view showing a protective device according to a preferred embodiment of the invention being worn, the device incorporating any one of the orthoses;

FIG. 18 is a rear view showing the protective device of FIG. 17 being worn;

FIG. 19 shows the protective device of FIG. 17 in use to protect the wearer from seatbelt injury;

FIG. 20 shows the protective device of FIG. 17 in use to protect the wearer from injury or discomfort when lying prone;

FIG. 21 is a rear view of the protective device of FIG. 17;

FIG. 22 is a front view showing a protective device according to another preferred embodiment of the invention being worn, the device incorporating an orthosis;

FIG. 23 is a front view showing a protective device according to a further preferred embodiment of the invention in use to protect a wearer thereof from seatbelt injury, the device incorporating an orthosis;

FIG. 24 is a front view showing a garment, incorporating an orthosis, according to a preferred embodiment of the invention being worn; and

FIG. 25 is a front view showing a protective device according to an additional preferred embodiment of the invention mounted to a seatbelt to protect a person from being injured by the seatbelt, the device incorporating an orthosis.

### DETAILED DESCRIPTION OF THE PREFERRED EMBODIMENTS OF THE INVENTION

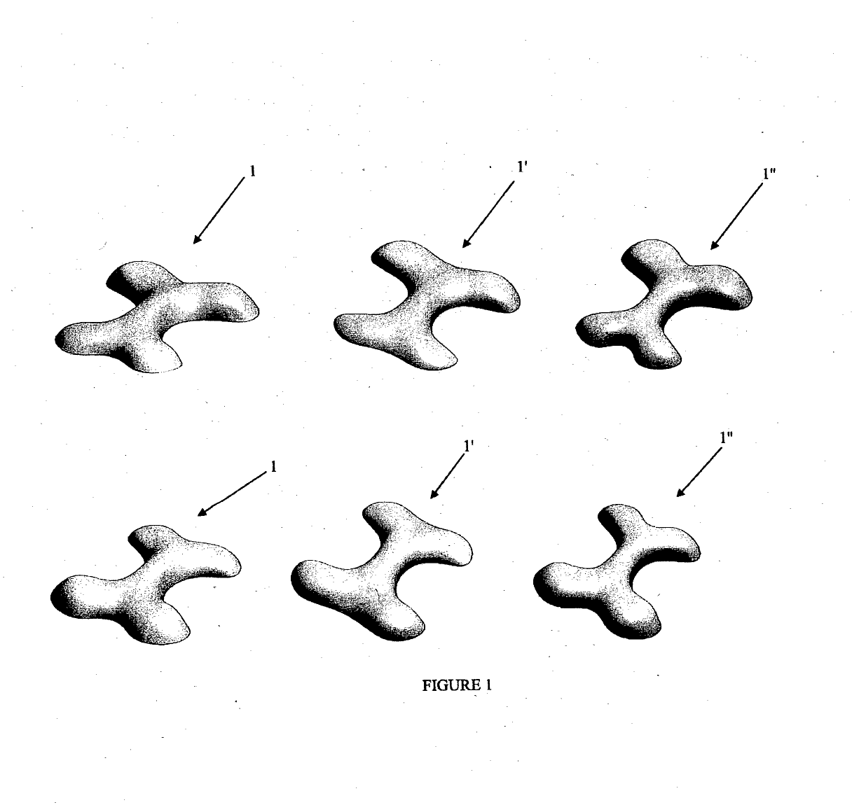

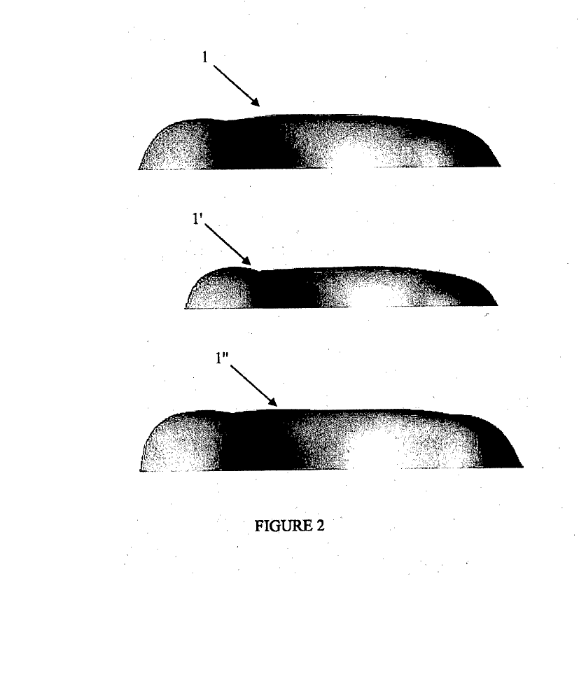

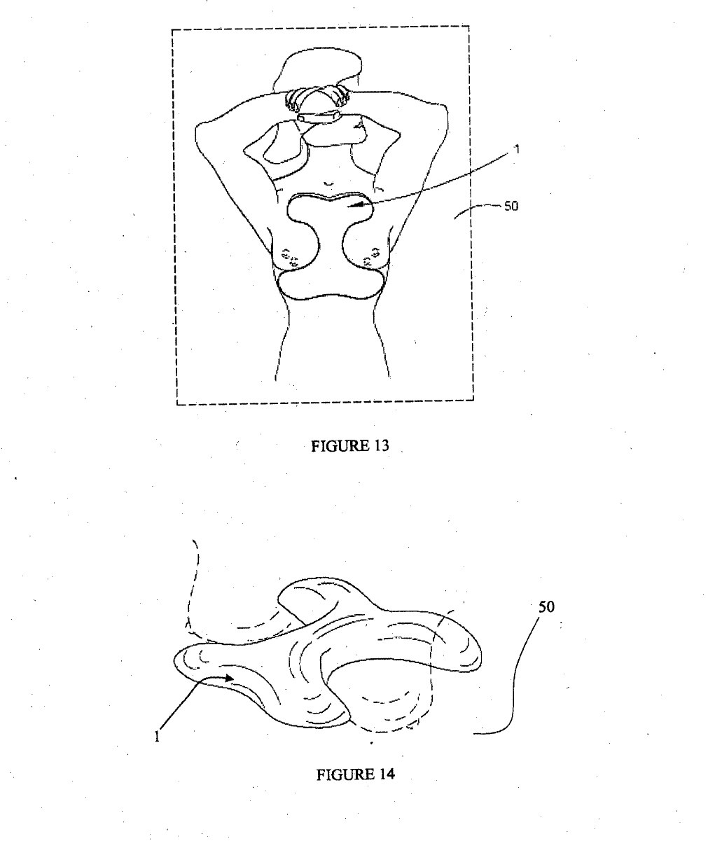

FIGS. 1 and 2 provide upper perspective views and side elevation views, respectively, of different-sized orthoses 1, 1′ and 1″ according to preferred embodiments of the present invention. Each of the orthoses 1, 1′ and 1″ is designed to overlie a support surface, with a base thereof received against the surface, to engage parts of the chest and upper abdomen area of a subject lying prone on the surface (see FIGS. 13 and 14) in a manner such that they are protected from unwanted compression, displacement or derangement. Each orthosis is used to direct compression and displacement pressures away from pain-sensitive breast tissue to more structurally robust tissues including the sternum, clavicle and rib cage and configured such that, when it is matched to the appropriate breast size, breast tissue, to the extent that it is displaced, is guided by the orthosis and is so displaced in the most favourable lateral and/or superolateral direction, unimpeded by any part of the orthosis, generally towards the anterior or auxiliary fold.

Generally speaking, the orthoses 1, 1′ and 1″ are designed for subjects having cup sizes C to D, A to C, and D to G respectively.

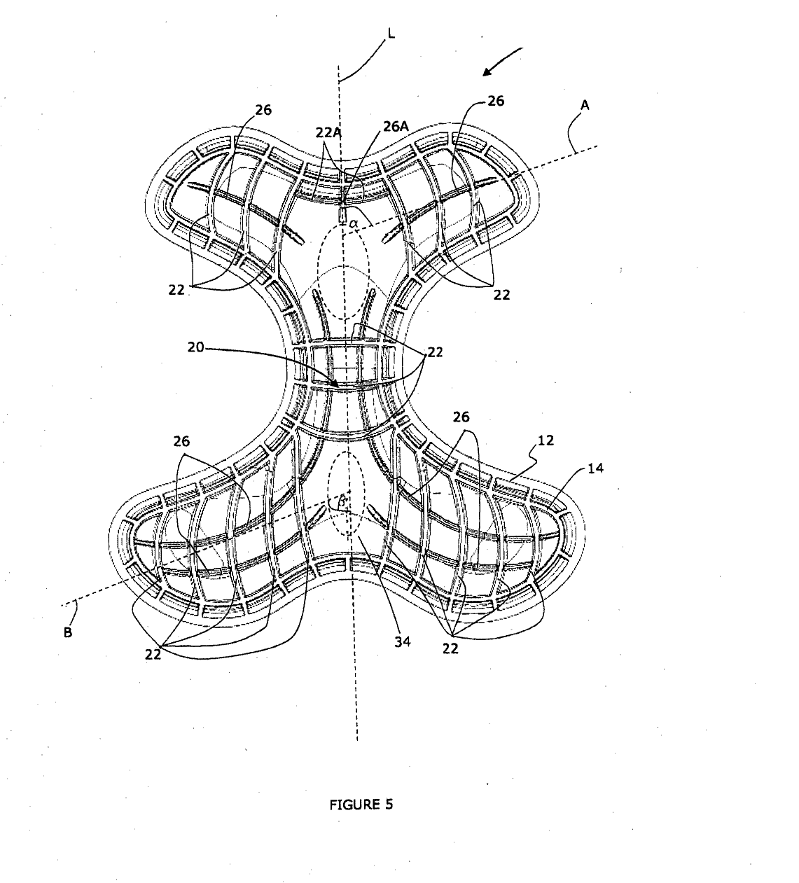

Each of the orthoses 1, 1′ and 1″ is configured, upward of its base, with an exterior shape which is substantially identical to that of a respective one of correspondingly sized orthoses illustrated in FIGS. 5A and 5B of the specification of the inventor's international patent application publication no. WO2011/106836 (“the earlier specification”), and described in that specification. However, each of the orthoses 1, 1′ and 1″ comprises a body which is hollow, rather than substantially solid, and is appropriately stiffened via ribbing. Advantageously, these orthoses can be more simply, quickly, cheaply and reliably manufactured, particularly (though not necessarily exclusively) by injection moulding. The quantity of material making up each orthosis is also considerably reduced, and thus so are associated material costs.

Each of the orthoses 1, 1′ and 1″ is injection moulded as a single piece from a thermoplastic elastomer, which is preferably medical grade and may comprise, for example, a thermoplastic elastomer marketed under the name Mediprene, which is a trade mark owned by VTC Elastoteknik AB Company Sweden, or a thermoplastic elastomer marketed under the name Thermolast, which is a trade mark owned by Kraiburg TPE GmbH and Co. KG, and more particularly one which is referred to as Thermolast-M. Accordingly, each orthosis is particularly well suited for contact with the skin. Moreover, each orthosis is durable, steam/autoclave sterilisable, cleanable with alcohol, washable (including by a dishwasher). Each orthosis is thus suitable for multiple-use, including from one person to another without risk of transfer of infection, and thus for clinical applications.

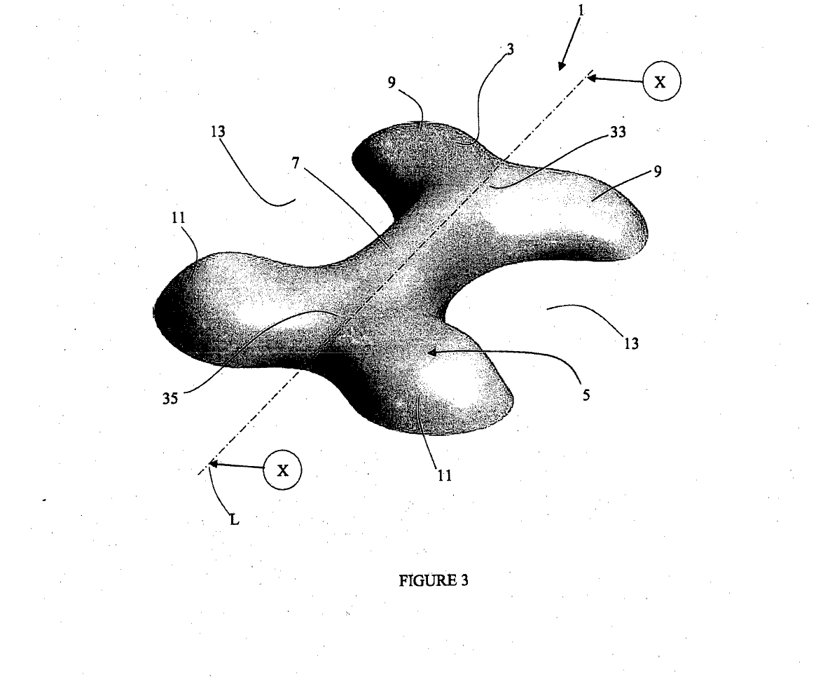

The orthosis 1 according to the first preferred embodiment of the present invention, referring in particular to FIGS. 3, 5 and 6, comprises a continuous exterior wall 3 which is of substantially uniform thickness, that thickness preferably being approximately 7 mm, the wall 3 defining a generally hollow body 5 of the orthosis 1. The body 3 is open at an underside/base of the orthosis 1, thereby defining a downwardly opening cavity 6 which is surrounded by the wall 3.

The orthosis 1 comprises a central portion 7 which extends along a central cephalad-caudal axis L of the orthosis so as to be receivable between the breasts of the subject to support the sternum and to line up therewith at the midline of the body, opposed portions 9 extending bilaterally outwardly from a cephalad end of the central portion 7 to support the clavicle and/or upper rib cage/costal cartilage (“cephalad portions”) and opposed portions 11 extending bilaterally outwardly from a caudal end of the central portion 7 to support the lower rib cage/costal cartilage and/or upper abdomen (“caudal portions”). The orthosis 1 is symmetrical about a plane which extends through the axis L and perpendicular to the plane in which a base 8 of the orthosis 1 lies. The exteriors of the portions 7, 9 and 11 upward of the base 8 are defined in their entirety by the wall 3/body 5.

The wall 3/body 5 is contoured for comfort and to avoid pinching of tissue when in use. Accordingly, the orthosis is conveniently shaped so that, inter alia, the subject-contacting surface portions thereof are smoothly curved and sloped to contact breast tissue comfortably and gently when the orthosis 1 is in use. The body 5 has a generally I-shaped configuration, opening bilaterally outwardly of the central portion 7, whereby spaces 13 are defined laterally outward of the axis L, which spaces are arranged to be occupied by the breasts of the subject when prone on the support surface and supported by the orthosis 1, as will be described in further detail later. The orthosis 1 deforms resiliently when engaged by the subject, thereby supporting the subject in a cushioning manner, as will also be described in further detail later.

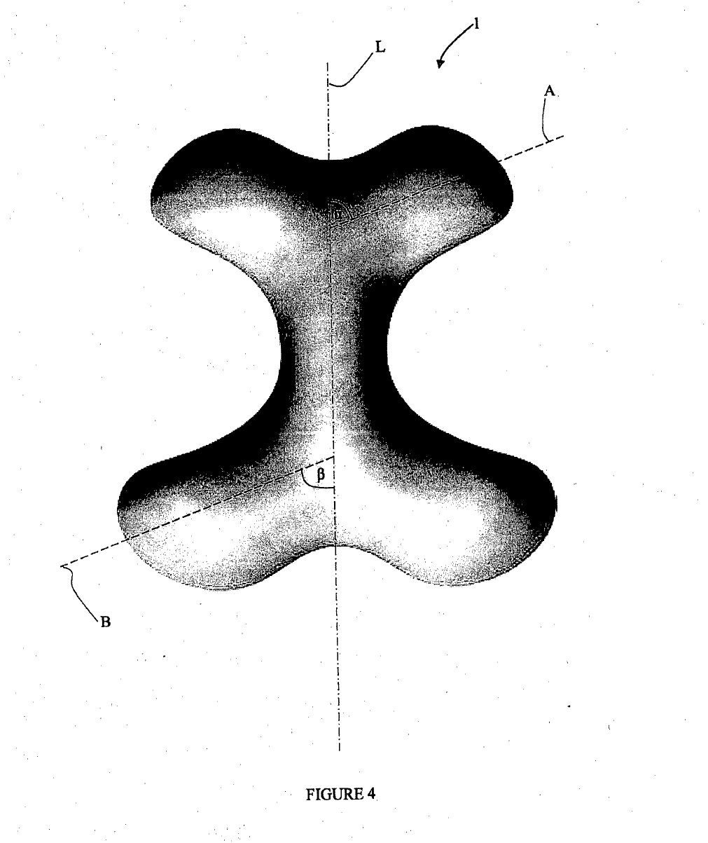

Referring to FIGS. 4 and 5, each cephalad portion 9 extends from the central portion 7 along a respective generally central axis A at an angle α of approximately 60 degrees to the axis L. Similarly, each of the caudal portions 11 extends along a respective axis B at an angle β of about 75 degrees to the axis L. Respective exterior surface portions of the wall 3 are arranged to contact the subject generally along the axes A, B and L and each of those surface portions when viewed in section transverse to the respective axis is upwardly convex.

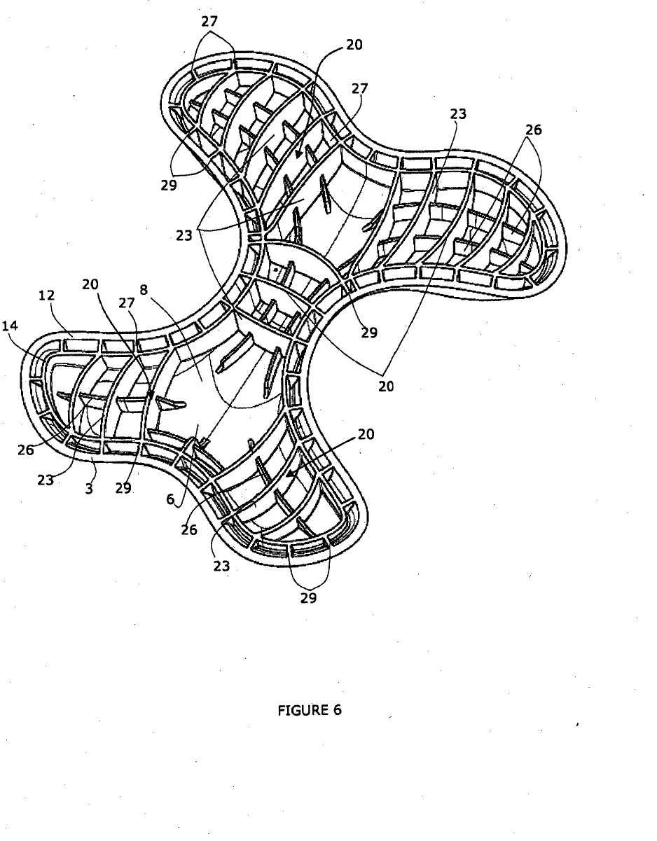

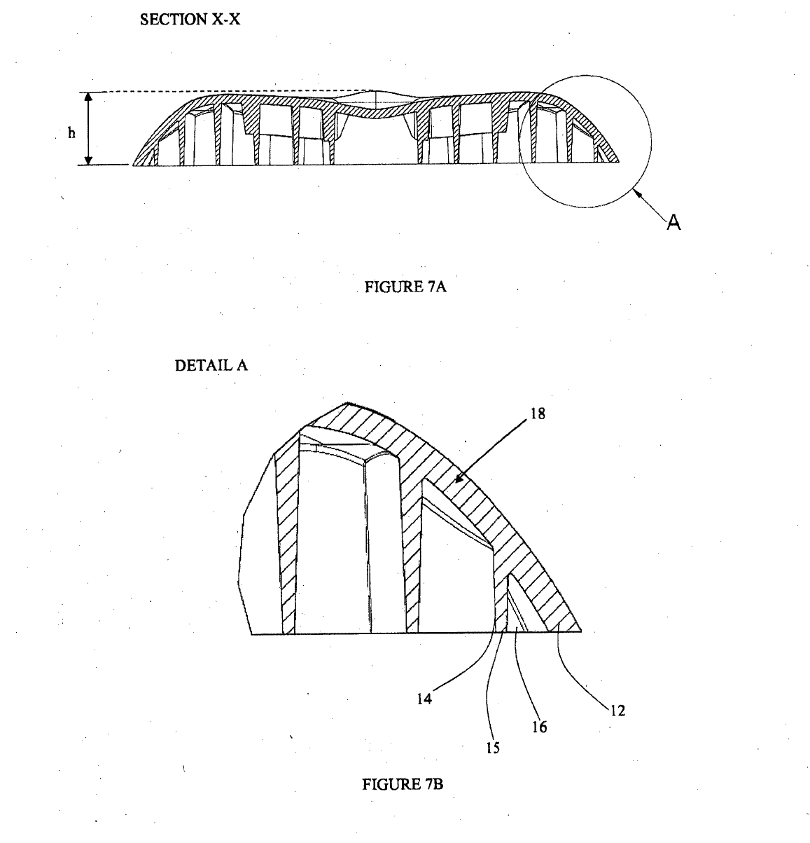

Referring to FIGS. 5 and 6, the orthosis 1 additionally comprises a resiliently deformable rib structure 20 which provides stiffening to the wall 3 such that it deforms appropriately when engaged by the subject. The rib structure 20 comprises respective groups of ribs forming part of the central portion 7, cephalad portions 9 and caudal portions 11. Each group of ribs comprises transverse ribs 22 and one or more longitudinal ribs 26 interconnected with those transverse ribs 22 to stabilise them. Each of the transverse ribs 22 is integrally formed with the wall 3. Exposed (lowermost) edges 23 of the ribs 22 lie in the same plane as the continuous peripheral edge 12 of the wall 3/body 5, whereby the edges 23 and edge 12 contact a satisfactorily flat area of a support surface, on which the subject using the support is to lie. The longitudinal ribs 26, although they adjoin the wall 3 along the entireties of their lengths (consistent with the ribs 22), do not extend to the plane of the base 8 but rather extend part way to the base, and roughly a third to a half of the depth of the respective sections of the cavity 6 at which they are positioned. Each of the longitudinal ribs 26 provides bracing to the transverse ribs 22 with which it is interconnected, thereby appropriately limiting the degree to each of the transverse ribs 22 is displaced/deformed under load. The ribs 22 and 26 in each of the sections 7, 9 and 11 define a respective frame structure, in the form of a grid or lattice, in that section.

Projecting downwardly from an interior face of the wall 3 is a rib 14 an exposed (lowermost) continuous edge 15 of which is spaced radially inward a substantially uniform distance from the edge 12, the edge 15 extending generally parallel to, and being coplanar with the edge 12. Short ribs 27 are arranged at approximately regular intervals around the rib 14 and extend radially across a gap 16 between rib 14 and a peripheral edge portion 18 of wall 3/body 5, so as to interconnect the rib 14 and edge portion 18. Exposed (lowermost) edges 29 of the ribs 27 are also coplanar with the edge 12; the base 8 of the orthosis 1 is thus defined by the edges 12, 15, 23 and 27, which all lie in a common plane to contact the flat surface against which the base 8 rests. The edge portion 18, rib 14 and ribs 29 form a peripheral seating portion of the orthosis 1 which can, advantageously, remain in contact with the flat surface when the orthosis 1 is loaded, owing to a degree of relative movement between the edges 12 and 15 being permitted.

Each of the ribs 22 adjoins, along the entirety of its periphery contained within the cavity 6, the wall 3 and rib 14.

Each of the ribs 14, 22, 26 and 29 is of gradually reducing thickness down its depth, so as to be configured with a draft, permitting removal of the orthosis from an injection mould. Each of the transverse ribs 22 and longitudinal ribs 26 has a thickness of about 5 mm at an upper end thereof and a thickness of about 3 mm at a lower end thereof. The continuous rib 14 has a thickness of about 4 mm at an upper end thereof and 3 mm at a lower end thereof. Each radial rib 27 has a thickness of 3 mm at an upper end thereof and 2 mm at a lower end thereof.

The orthosis 1 additionally includes, at a cephalad end thereof, a short longitudinal rib 26A extending, along the axis L, from the continuous rib 14 towards the caudal end of the orthosis 1, and a pair of short transverse ribs 22A each extending from the rib 26A to a respective one of the proximal-most transverse ribs 22 of the cephalad portions.

The transverse ribs 22 of the central portion 7 extend transverse to the axis L, each of those ribs spanning the respective section of the cavity 6 which it occupies. In the present embodiment, the number of those ribs is three (though this number may vary without departure from the invention). Each of those ribs is bowed in a direction along axis L so as to influence appropriately the manner in which the central portion 7 deforms when the subject is received against the orthosis 1. More particularly, a first one of those ribs which is disposed at or adjacent to a caudal end of the central portion 7 is bowed in the caudal direction along axis L, a second one of those ribs, which is closer to a transverse axis T through the narrowest section of the central portion 7 (which axis is perpendicular to axis L) is also bowed, but to a lesser extent, in the same direction, and a third one of those ribs, which is disposed on the opposite side of the axis T to the second rib, is bowed in a cephalad direction along axis L. All of these ribs are bowed symmetrically about axis L. The degree to which each rib 22 is bowed is generally commensurate with its distance, along axis L, from axis T. Each central portion transverse rib 22 bows in the same direction as that in which it is spaced from the axis T. The second (middle) transverse rib of the central portion 7 can be omitted without departure from the invention, if required having regard to anatomical/comfort considerations, whereby the region in which that rib is disposed will be of reduced stiffness.

The central portion has a pair of longitudinal ribs 26 which are arranged to either side of the axis L and are bowed laterally inwardly, having curvatures generally following those of the laterally outermost edges of the central portion. Cephalad ends of those ribs 26 are tapered whereby tips at those ends are flush with the interior face of wall 3. In an alternative embodiment of the invention, the central portion may, for example, have a single longitudinal rib 26 which is centred over and extends in alignment with the longitudinal axis L and is axially outwardly tapered at its ends such that tips at those ends are flush with the interior face of wall 3.

The transverse ribs 22 in each cephalad portion 9 consist of three such ribs, which bow towards a distal end of the portion 9 to a progressively increasing extent from the first to the final rib in that direction, so as to influence appropriately the manner in which the cephalad portion 9 deforms when the subject is received against the orthosis 1. The second rib 26 in that direction (middle rib) can be omitted without departure from the invention, if required having regard to anatomical/comfort considerations, whereby the region in which that rib is disposed will be of reduced stiffness. Each cephalad portion 9 comprises a single respective longitudinal rib 26 which is generally centred in the section of the cavity 6 along which it extends. Opposite ends of each caudal portion longitudinal rib 26 are tapered axially outwardly to form tips, axially outermost extents of which coincident with the interior face of the wall 3.

Each of the caudal portions 11 includes five transverse ribs 22 which bow towards a distal end of the portion 11 to progressively increasing extents in the direction from the first to the final rib in that direction, so as to influence appropriately the manner in which the caudal portion 11 deforms when the subject is received against the orthosis 1. The second and fourth ribs 22 in that direction can, without departure from the invention, be omitted if required having regard to anatomical/comfort considerations, whereby the regions of the portion 11 in which those ribs are disposed will be of reduced stiffness. Each caudal portion 11 comprises a pair of longitudinal ribs 26, which ribs are transversely spaced apart. The axially outer (caudal-most) one of longitudinal ribs 26 terminates at the distal-most transverse rib 22 whereas the axially inner (cephalad-most) one of those longitudinal ribs 26 terminates at the edge portion 12 at the distal end of the respective caudal portion 11. Proximal ends of the caudal-most ribs 26 are tapered in the same manner as the longitudinal rib ends as previously described. Each of the cephalad-most ribs 26 is formed so as to be integral or continuous with a respective one of the ribs 26 of the central portion.

The longitudinal ribs 26 in the cephalad portions 9 and caudal portions 11 are bowed so as generally to follow, respectively, the curvatures of those portions.

The bowing of the ribs 22 and 26 (in a plane parallel to the plane of the base 8) predisposes them to flexure in the directions in which they are bowed, as will be described in further detail shortly.

An absence of ribbing at a cephalad end region of the central portion 7 and proximal end regions of the cephalad portions 9 confers a reduced stiffness to a section 32 of the wall 3/orthosis 1 occupying those regions, whereby that section deforms sufficiently when loaded so as not to exert undue pressure on the manubrio-sternal junction, which could cause discomfort. Moreover, the exterior of the section 32 is preferably configured with a concavity or “relief” 33, which accommodates that junction.

Also, the absences of a transverse rib in a region of the central portion 7 which is caudal of the aforementioned first rib 22 along axis L and of ribs at proximal end regions of the caudal portions confer a reduced stiffness to a section 34 of the wall 3/orthosis 1 occupying those regions, whereby that section deforms sufficiently when loaded so as not to exert undue pressure on, or cause excessive displacement of, the xyphoid process, which could also cause discomfort. Moreover, the exterior of the section 34 is preferably configured with a concavity or “relief” 35, which accommodates the xyphoid process.

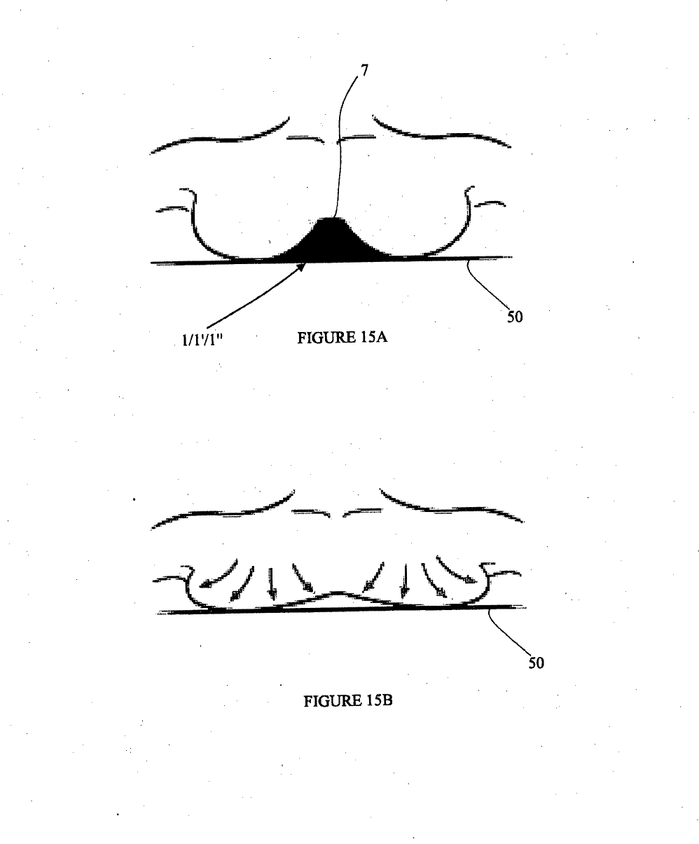

Use of the orthosis 1 will now be described with reference to FIGS. 13, 14, 15A, 15A, 16A and 16B.

Referring firstly to FIGS. 13 and 14, with the orthosis 1 resting with its base 8 in contact with support surface 50, which may be provided by, for example, a therapy table, mattress or yoga mat, a woman assumes a prone lying position on the surface 50 such that the central portion 7 is received between her breasts to support her sternum, the cephalad portions are received against and support her clavicle and/or upper ribcage/costal cartilage, and the caudal portions are received against and support her lower ribcage/costal cartilage and/or upper abdomen, whereby pressure on, and undesirable displacement of, breast tissue are minimised. To the extent that the breasts are displaced as a result of the engagement between them and the orthosis 1, they are displaced laterally or superolaterally, unimpeded by any part of the orthosis 1 and in a manner controlled by the orthosis 1, generally towards the anterior or auxiliary fold and such that they are received by the spaces 13.

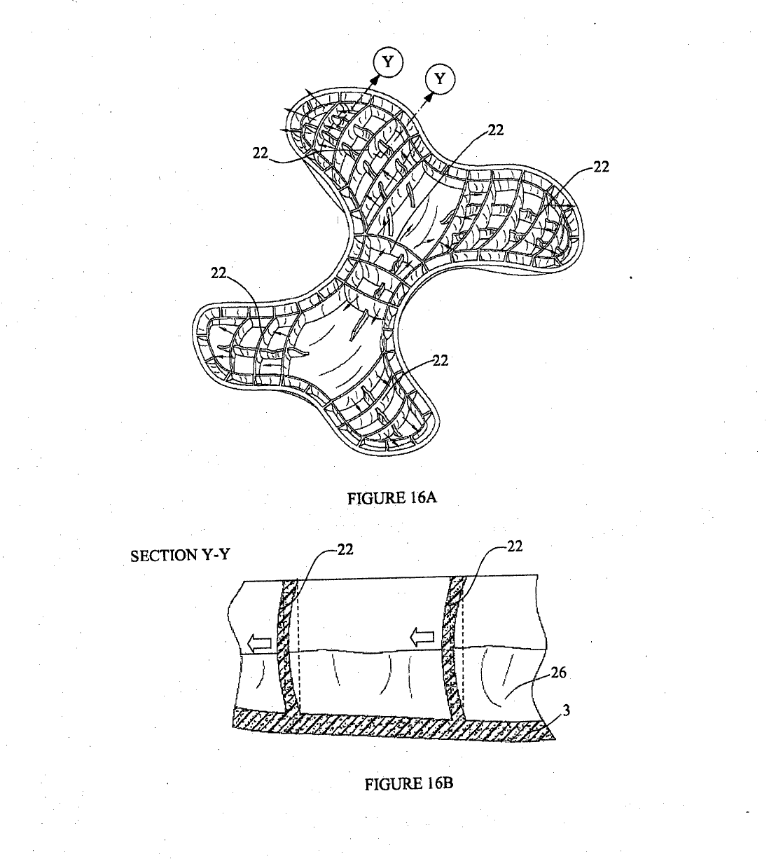

Referring now to FIGS. 16A and 16B, the latter showing section Y-Y as indicated in the former (the deformation details of which section are typical for the transverse ribs generally), engagement between the woman and the orthosis 1 causes the transverse ribs 22 to flex in the respective directions in which they are bowed, the flexure being in a plane which is generally orthogonal to the respective rib at the position at occurs, i.e. generally radial with respect to the ribs 22, whereby the ribs 22 function as springs. The longitudinal ribs 26 preclude control the deformation of the transverse ribs 22 with which they are connected, precluding the transverse ribs 22 from collapsing.

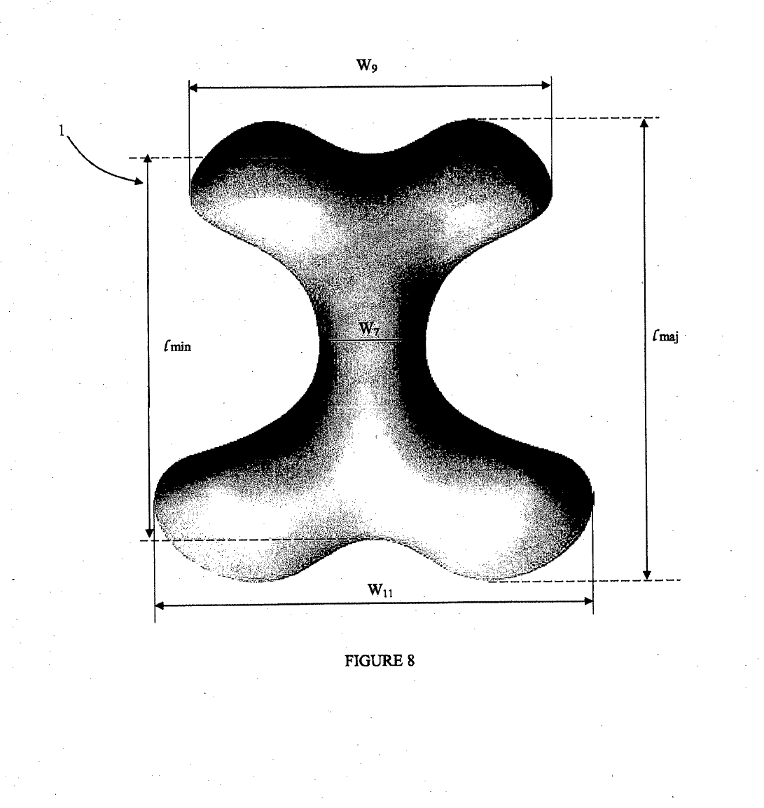

The orthosis 1 according to the present embodiment is medium-size, and, generally speaking, suitable for bra cup sizes D to E. Referring to FIG. 8, the orthosis 1 has a major length dimension lₘₐⱼ, between transverse axes passing through axially outermost extents of the caudal 11 and cephalad 9 portions, of approximately 32 cm to 33 cm, a minor length dimension lₘᵢₙ, along the axis L, of approximately 25 cm to 26 cm, a width dimension w₇ across the narrowest section of the central portion 7 of about 7 cm, a minimum width dimension w₉ between distal ends of the cephalad portions 9 of about 24 cm and a maximum width dimension w₁₁ between distal ends of the caudal portions 11 of about 20 cm. The orthosis 1 has a peak height h of 4.5 cm, which is located approximately at the aforementioned narrowest section of the central portion 7.

In the description and drawings of the orthoses 1′ and 1″ of the further illustrated embodiments in the invention, which are intended for a subject of the same general size as, but having breasts which are smaller and larger, respectively, than those of the subject for whom the orthosis 1 is intended, the same reference numerals as have been used in respect of the orthosis 1 will be used to denote and refer to the same or corresponding features.

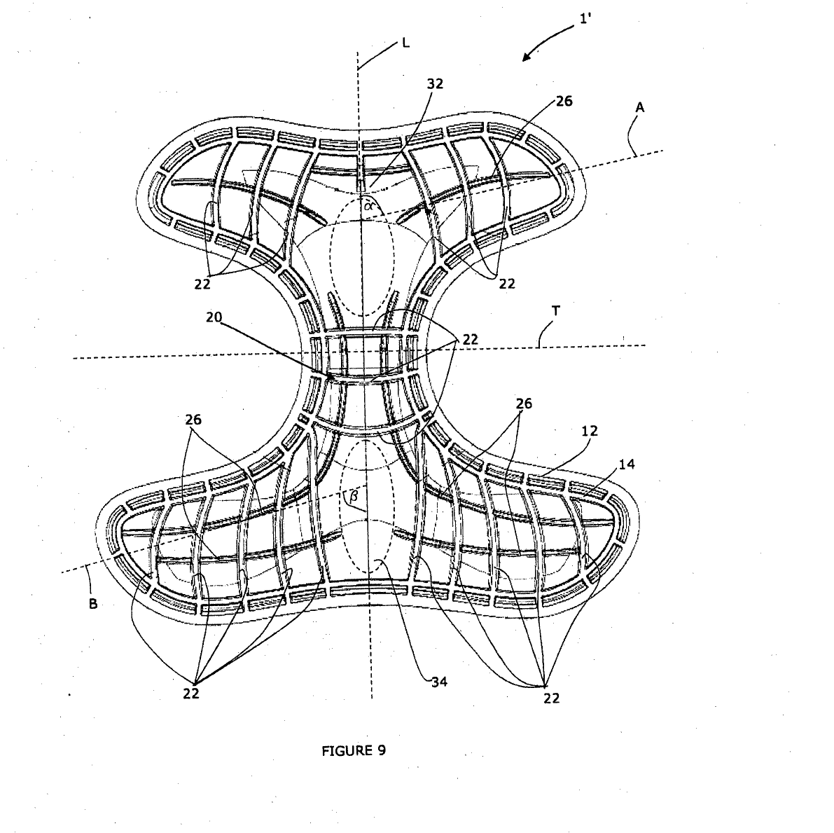

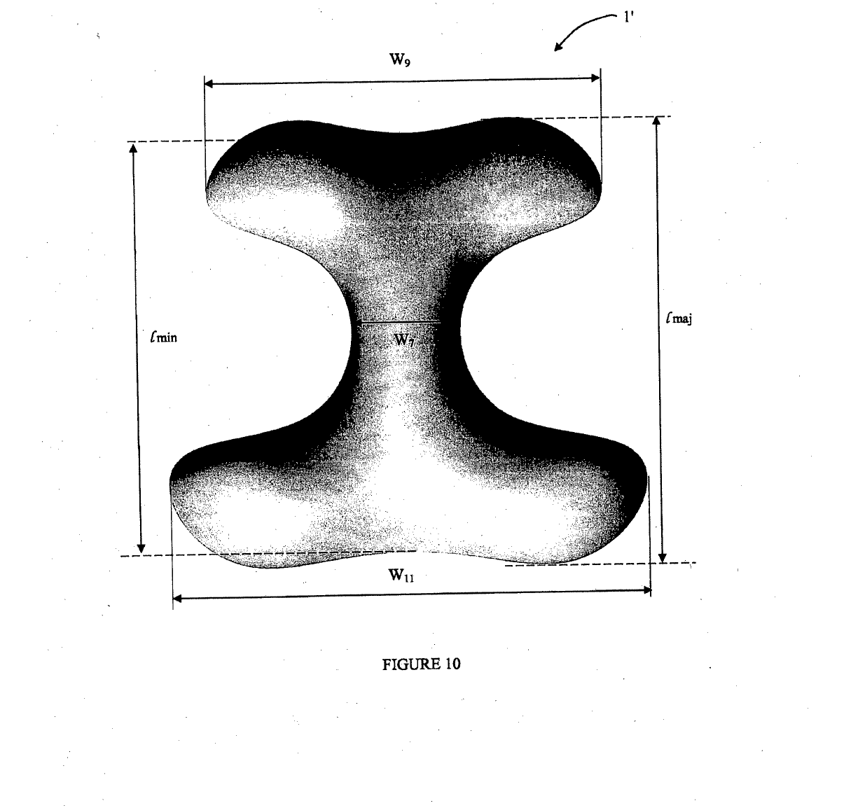

The small-size orthosis 1′ according to the second preferred embodiment of the present invention, which, generally speaking, is suitable for bra cup sizes A to C, is illustrated in FIGS. 9 and 10. The orthosis 1′, though similar to orthosis 1, incorporating a rib structure 20 substantially consistent with that in the orthosis 1, has a peak height of a centimetre or so less than that of the orthosis 1 and a wider central portion 7. More particularly, the orthosis 1′ has dimensions lₘₐⱼ, lₘᵢₙ, w₇, w₉, w₁₁ and h of approximately 26 cm, 24 cm to 25 cm, 6.5 cm, 23 cm to 24 cm, 28 cm and 33.5 cm respectively. The angles α and β which the axes A and B respectively form with the axis L are approximately the same as in the orthosis 1. The peak height is generally at the same location as the dimension w₇, consistent with the orthosis 1. The spaces 13 in the orthosis 1′ are slightly further apart than in the orthosis 1 and laterally inwardly bounded by curved edge portions of the body 3 which have a smaller radius of curvature than in the orthosis 1.

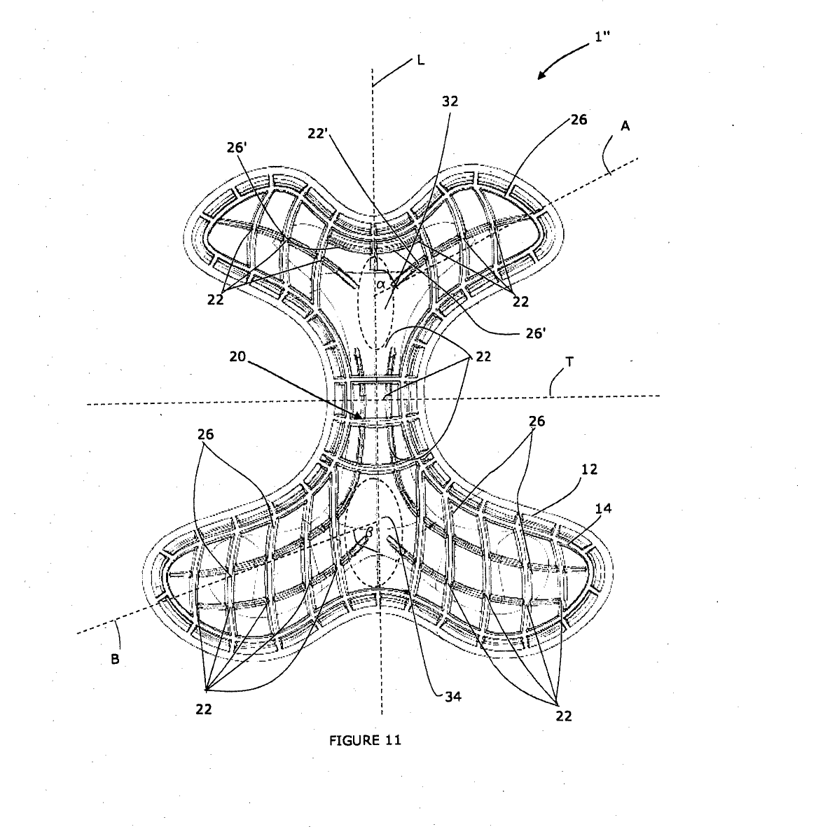

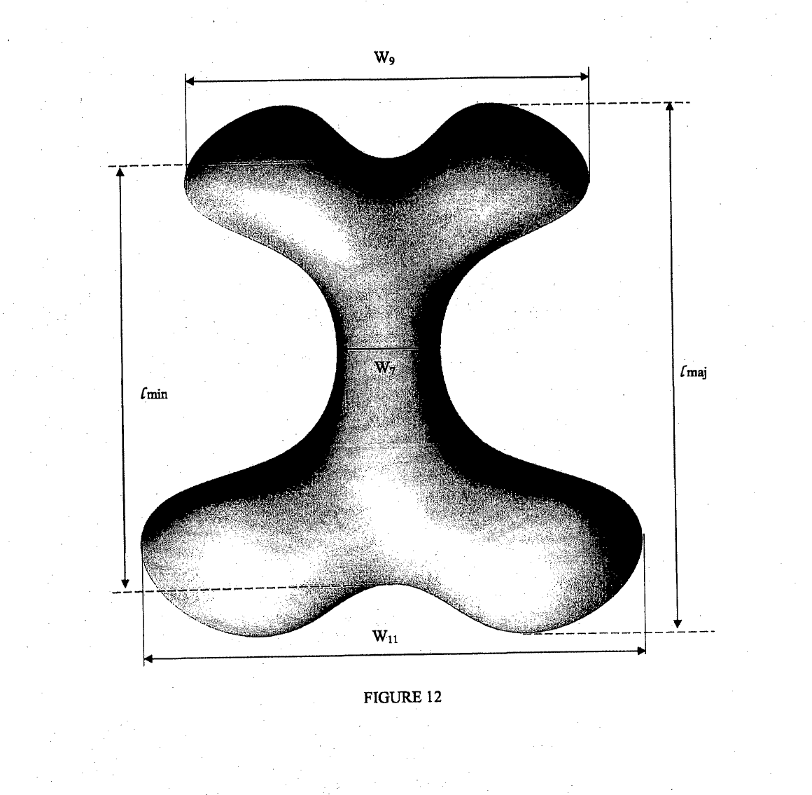

The large-size orthosis 1″ according to the third preferred embodiment of the present invention, which is suitable for bra cup sizes EE an up, is illustrated in FIGS. 11 and 12. The orthosis 1″, though similar to orthosis 1, has a peak height of a centimetre or so more than that of the orthosis 1 and a narrower central portion 7. More particularly, the orthosis 1″ has dimensions lₘₐⱼ, lₘᵢₙ, w₇, w₉, w₁₁ and h of approximately 32 cm, 26 cm, 62 cm to 63 cm, 25 cm, 28 cm and 4.5 cm to 5 cm respectively. The angles α and β which the axes A and B respectively form with the axis L are approximately the same as in the orthosis 1. The peak height is generally at the same location as the dimension w₇, consistent with the orthosis 1. The spaces 13 in the orthosis 1″ are slightly closer together than in the orthosis 1 and laterally inwardly bounded by curved edge portions of the body 3 which have a larger radius of curvature than in the orthosis 1. In the orthosis 1″, the difference between the dimensions lₘᵢₙ and lₘₐⱼ is greater than in the orthoses 1 and 1′, to accommodate the subject's neck. The orthosis 1″ also has a rib structure 20 which is substantially consistent with that in the orthosis 1 but which includes supplementary ribbing arranged at proximal ends of the cephalad portions 9/a cephalad end of the central portion 7, adjacent the continuous rib 14, to stiffen the orthosis 1″ adequately in the region of the resulting more pronounced “dip” created as a result of the aforementioned difference in the dimensions lₘᵢₙ and lₘₐⱼ. The supplementary ribbing comprises a rib 22′ which aligns with/lies on the axis L, that rib being transverse with respect to the cephalad portions 9 (but longitudinal with respect to the central portion 7) and serving as a rib for both portions 9, and ribs 26′ which are longitudinal with respect to the cephalad portions 9 (but define a single rib that is transverse with respect to the central portion 7) and join the rib 22′ at laterally inner ends thereof. The rib 22′ connects at a cephalad end thereof to rib 14 and is tapered at a caudal end thereof in the same manner as described previously in respect of the longitudinal ribs 26. The rib 22′, like the other transverse ribs 22 is anchored around the entirety of the periphery thereof which lies in the cavity 6, and has a (lowermost) edge which lies in the plane of, and thus forms part of, the base 8. The rib 26′, like the other longitudinal ribs 26, adjoins an interior face of the wall 3/body 5 and depends therefrom, and extends about a third to about half of the cavity depth, terminating at proximally innermost ones of the transverse ribs 22 of the respective cephalad portions 9.

The orthoses 1′ and 1″ deform in substantially the same manner as the orthosis 1 in use, and FIG. 13 onwards herein are thus generally applicable to all of the embodiments 1, 1′ and 1″.

The thicknesses of the wall and ribs in the orthoses 1′ and 1″ are substantially the same as in the orthosis 1. The ribs have draft angles of about 2°, whereby they are of progressively reducing thickness towards their lower/exposed edges, permitting removal of the orthoses from the respective moulds, as will be understood by a person skilled in the art.

The provision of ribbing promotes/contributes to ease and reliability of moulding, and more particularly minimises/eliminates voids, reduces/minimises cycle time, promotes/ensures even flow of the injected material and reduces/minimises sink marks, as does the provision of the uniform, and relatively low, wall thickness. Were the structure which supports the body to comprise thick wall sections or solid blocks of material, instead of ribbing, these benefits would be realised to a lesser extent or not at all.

The stiffness and deformation characteristics of the orthosis depend, among other things, on the rib configuration and the hardness of the elastomeric material; less ribbing can, to an extent, be compensated for by more material hardness, and vice versa. For example, material having a Shore A hardness value of 30 may be appropriate in combination with the embodiment, described above, as including all of the transverse ribs shown in the drawings, whereas a Shore A hardness value of 40 may be appropriate where the intermediate transverse ribs are omitted as described above. Generally speaking, the Shore A hardness value can, be between about 10 and about 50, though it is often preferable that this value be between about 15 and about 30.

The orthoses 1, 1′ and 1″ bring about significantly reduced loading on, and displacement of, the breasts, consistent with the graphical representations and data provided in FIGS. 8 to 10 in the earlier specification and the associated description in that specification.

As described herein, the degree to which breast tissue (including natural tissue or implant-augmented tissue), when exposed to compressive loads such as during prone lying or spinal manipulation thrust, is compressed and undesirably displaced is substantially reduced owing to the orthosis. Compression loads may be derived from the weight of the subject's upper body or from loads applied to the upper body, typically the posterior aspects of the spine or ribcage during, for example, physical therapy or massage. The orthosis is resiliently deformable. The orthosis absorbs and redistributes compression loads away from pain-sensitive breast tissue onto less pain-sensitive parts of the body lying over the orthosis. The orthosis can control and restrict displacement of the breasts and reduce/eliminate stress on them, including compressive, torsional and shear stress within them, during prone lying, including during clinical examination or physical therapy.

The orthosis, without being bound to any particular mode of action, compared with prior art devices for breast positioning during procedures such as radiology, massage, and spinal manipulation, provides relative ease of use, and a greater capacity for safety, comfort and improved alignment and positioning, and allows for good exposure to the axillae and lymphatic system.

The orthoses 1, 1′ and 1″ are shown substantially to scale in FIGS. 1 to 6 and 8 to 12, though their dimensions and proportions may be varied, as may their hardnesses and ribbing configurations, to accommodate anthropometric variations and comfort requirements.

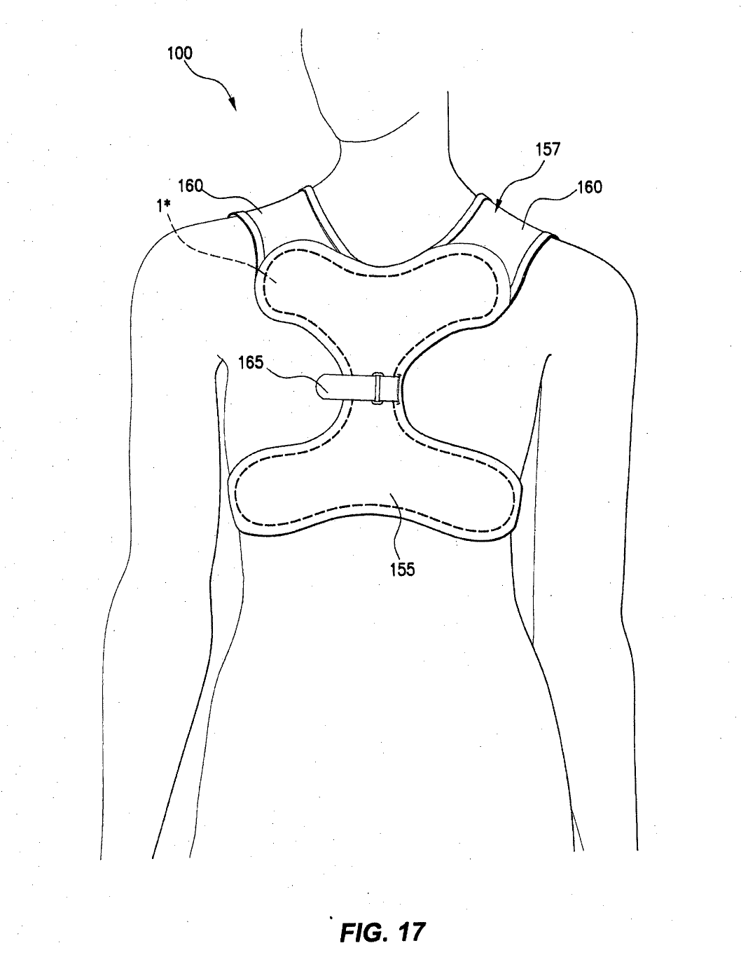

The invention is also embodied in protective devices, which will now be described with reference to FIGS. 17 to 25, each of which comprises attachment means configured to hold any one of the orthoses, 1, 1′ and 1″ (generally designated 1*) between the upper front torso of the person and an object (or objects) applying an external compressive load to the upper front torso, in a manner such that the orthosis 1* is received, directly or indirectly, against the subject whereby to abut the thoracic cage.

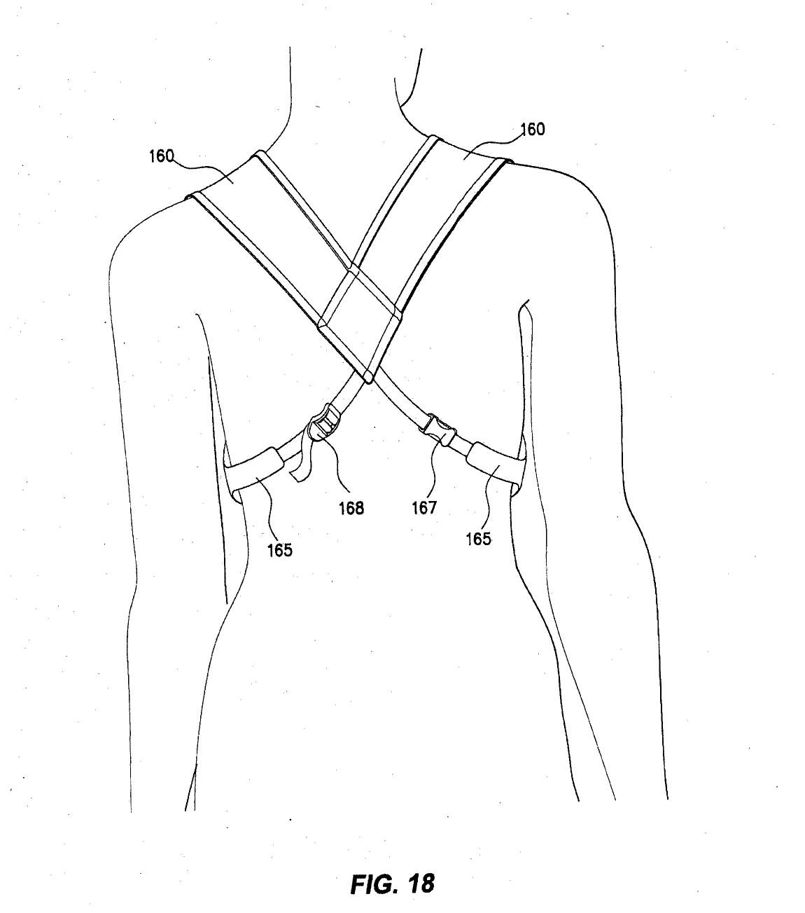

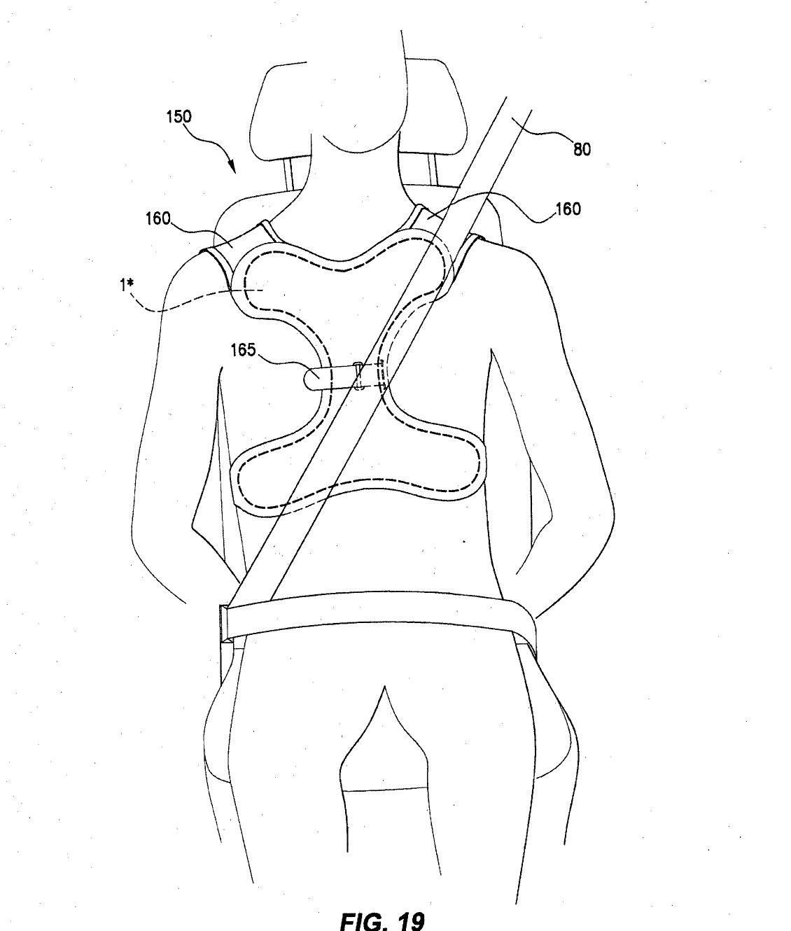

A protective device 100 according to one embodiment of the invention, which is illustrated at FIGS. 17 to 21, comprises a front panel 155, the orthosis 1* mounted against an inner side of the panel 155, and a light harness 157 connected to the panel 155. The periphery of the panel 155 is contoured so as to lie adjacent and follow generally the perimeter of the orthosis 1*, whereby the panel 155 is not obtrusive or cumbersome. The harness 157 comprises shoulder straps 160 which are arranged to extend over the shoulders and, in a converging manner, down the person's upper back, and length-adjustable side straps 165 which interconnect lower ends of the shoulder straps 160 with respective lower side portions of the panel 155. At least one of the straps 165 is provided with interengaging connectors 167 which are releasable such that the device 100 can be donned and doffed. At least one of the connectors 167 on the or each strap 165 can be manipulated to adjust the length of that strap, whereby the device 100 can be fitted to different-sized torsos. Where connectors 167 are provided on only one strap 165, an alternative length adjuster, such as a buckle-type adjuster 168 as can be seen in. FIG. 18. Various other harnessing/strapping arrangements are, of course, possible without departure from the invention, as will be known to a person skilled in the art.

The panel 155 is preferably flexible or resiliently flexible but is of a rigidity sufficient to distribute into the orthosis 1* a compressive load applied against the upper front torso by a support or restraint, which in the example shown comprises a seatbelt 80. The orthosis 1* referring to FIG. 21, is held against the panel 155 via elastic band members attached to the panel under respective ones of which the cephalad portions and caudal portions are received, and a releasable strap connector 165 which extends through laterally spaced apart slits formed through the panel 155 and around the central portion of the orthosis 1*. It will be appreciated that the orthosis 1* may be held to the inner side of the panel 155 in any of multiple other ways without departure from the invention.

Shown in FIG. 19 is the device 100 in use to protect the wearer's torso from soft-tissue injury (including but not limited to breast injury) caused by a seatbelt in the event of a collision or sudden deceleration. The device 100 may be configured such that diametrically opposite ones of the cephalad and caudal portions underlie the seatbelt 80, whereby there is direct transfer, through the orthosis 1*, of loading applied to the upper torso by the seatbelt 80.

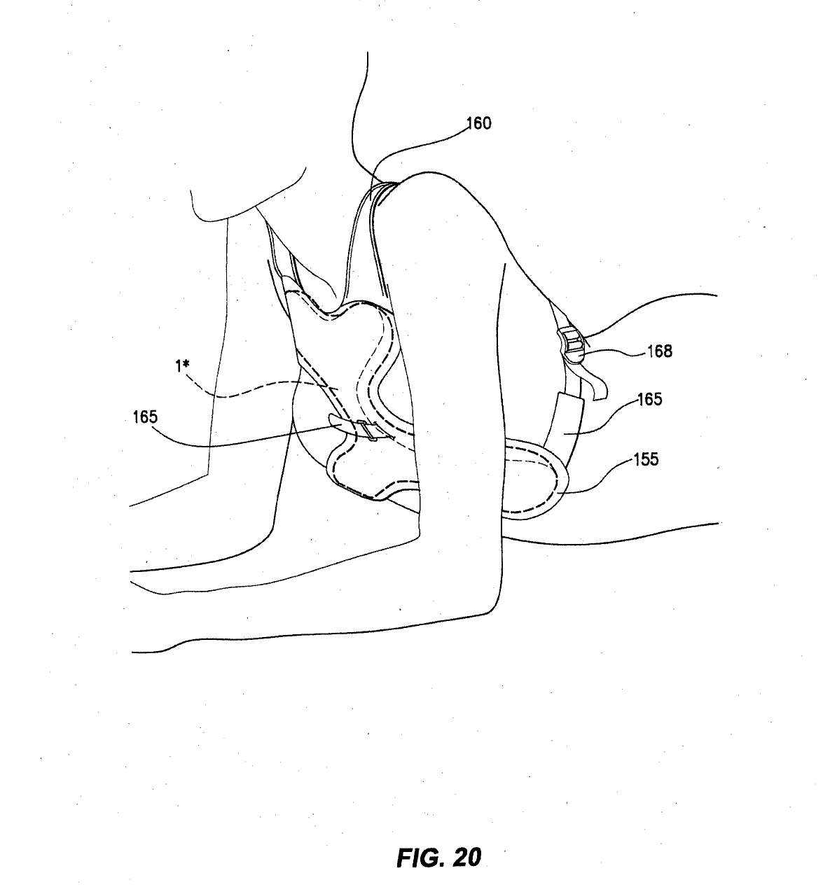

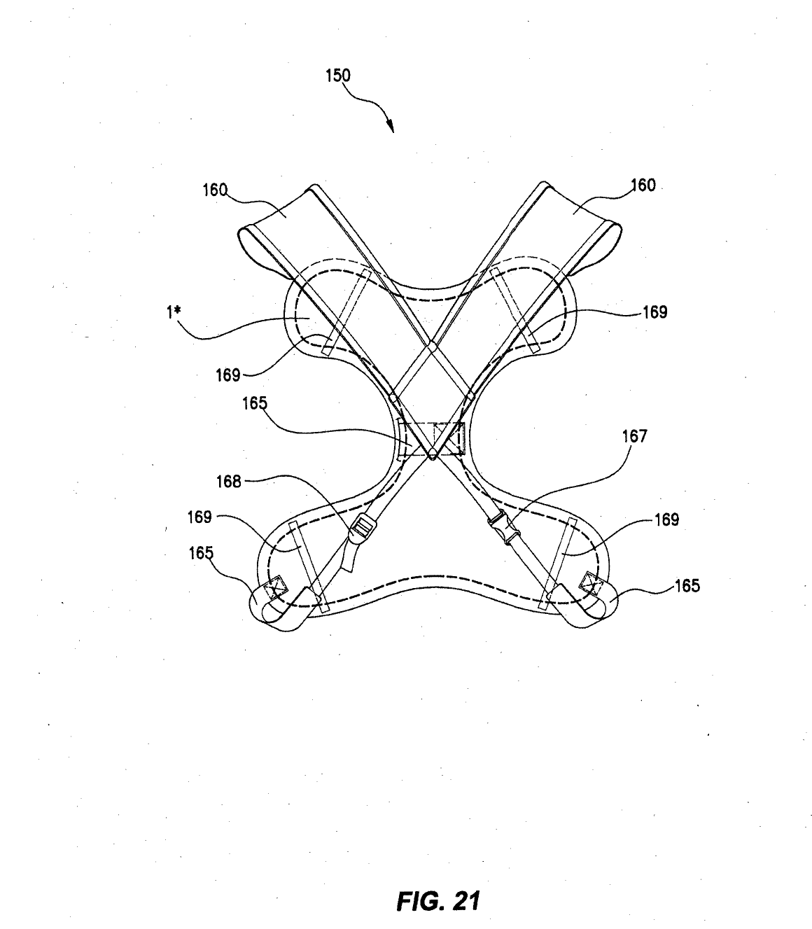

Shown in FIG. 20 is the device 100 being worn to protect the person while lying prone on a support surface, in a manner consistent with a use of the orthosis 1* on its own as previously described.

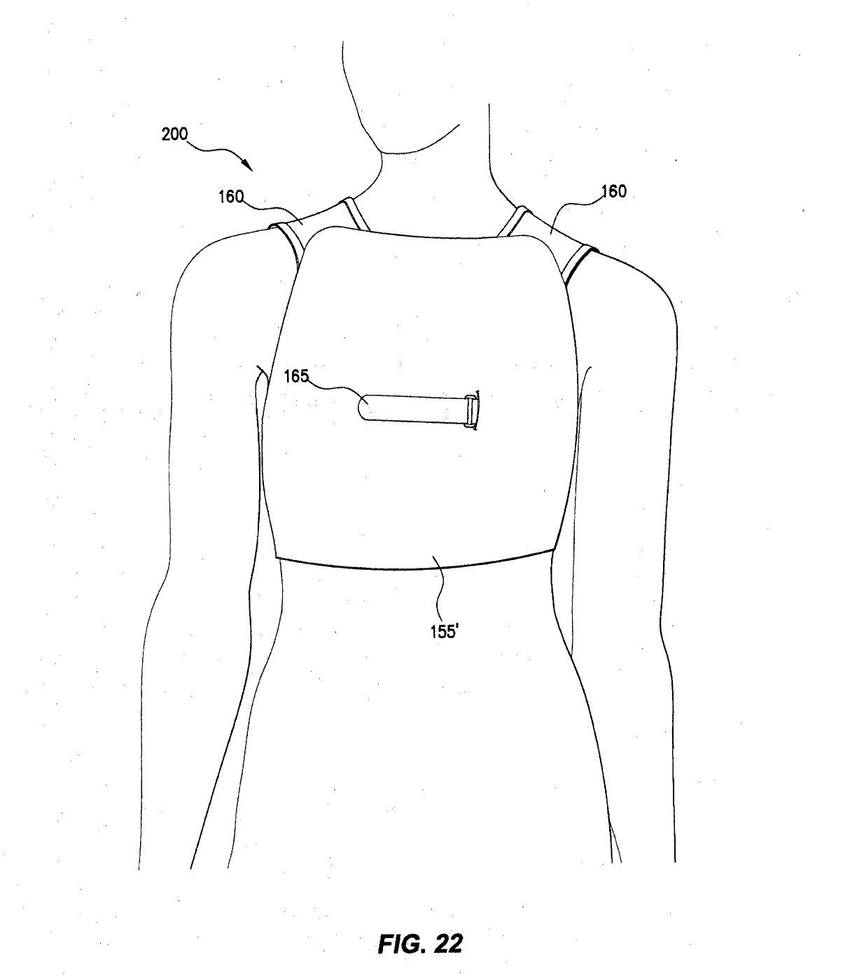

A protective device 200 according to another embodiment of the invention is illustrated at FIG. 22, and is substantially identical to the device 100 except that the front panel 155′ thereof is generally rectangular so as substantially to cover the upper torso. The panel's being so configured may promote/enhance more even or widespread distribution of the externally applied compressive load through the orthosis 1*.

It will be noted, having regard to the foregoing and drawings, that the protective device may take the form of a vest.

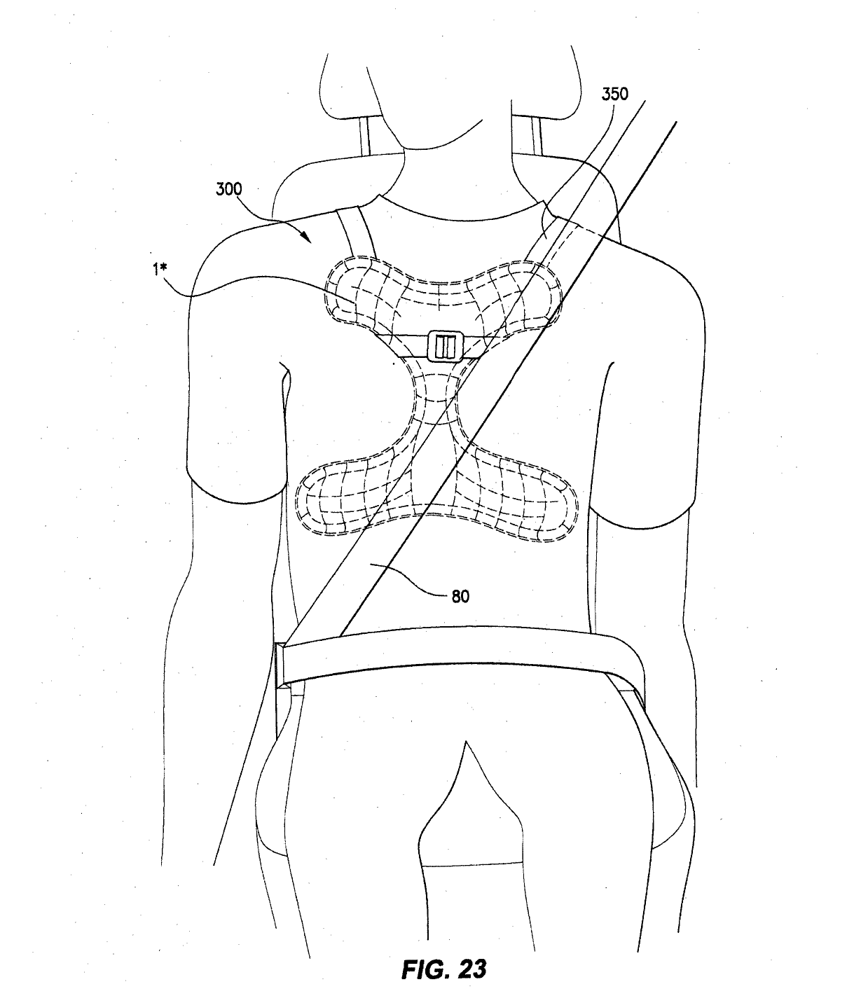

Shown in FIG. 23 is a protective device 300 according to another preferred embodiment of the invention. This device 300, instead of comprising a harness system, comprises a length-adjustable lanyard 350 which is received around the person's neck and engages the orthosis 1* such that it hangs from the neck to be positioned against the upper front torso adjacent the breasts. In the example shown, the lanyard 350 is trained under the cephalad portions and over an upper section of the central portion so as to hold the orthosis 1′. The lanyard may, in other examples, engage the orthosis 1* in any of multiple alternative manners without departure from the invention, as will be appreciated by a person skilled in the art.

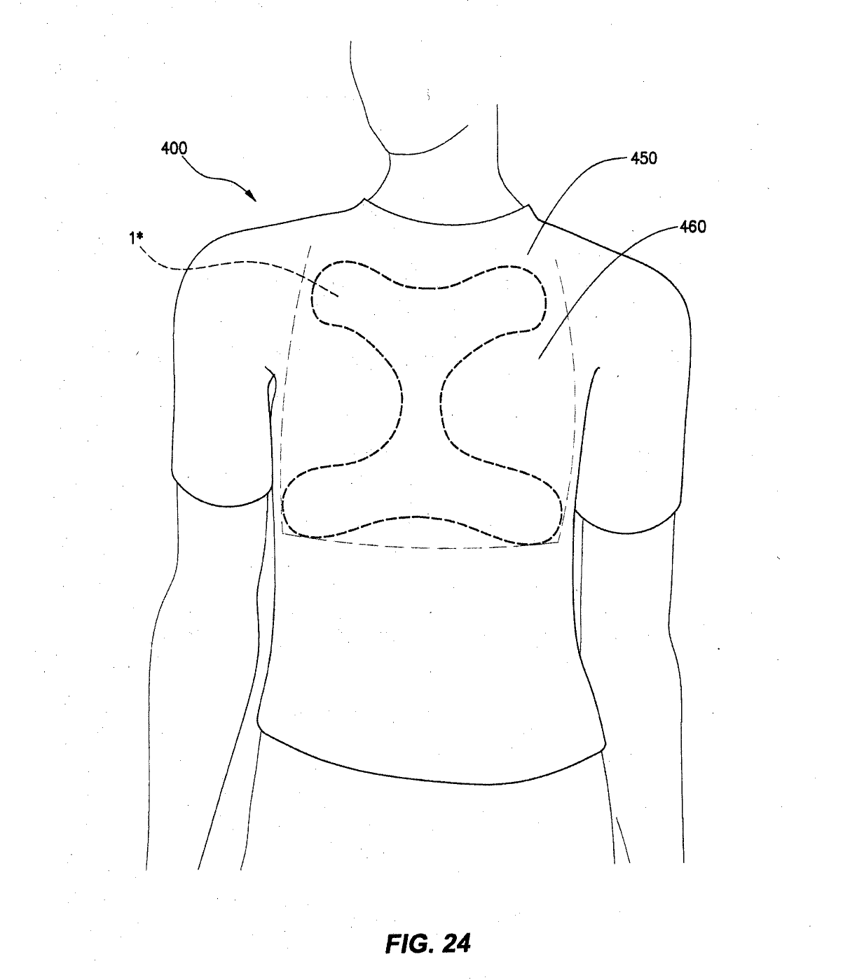

A protective device 400 according to yet another preferred embodiment of the invention is illustrated in FIG. 24. This device comprises a garment 450 which is worn on the person's torso and the orthosis 1* which is held by the garment 450. The garment in the example shown is a t-shirt but could instead be any other garment which is worn over the torso; e.g., a jumper, skivvy, vest or dress. The garment 450 is configured with a, preferably internal, pocket 460, pouch or other means for holding the orthosis 1* such that is received against the upper front torso when the garment is worn. It will be appreciated, again, that the means by which the orthosis 1 is held by the garment may be modified considerably without departure from the invention.

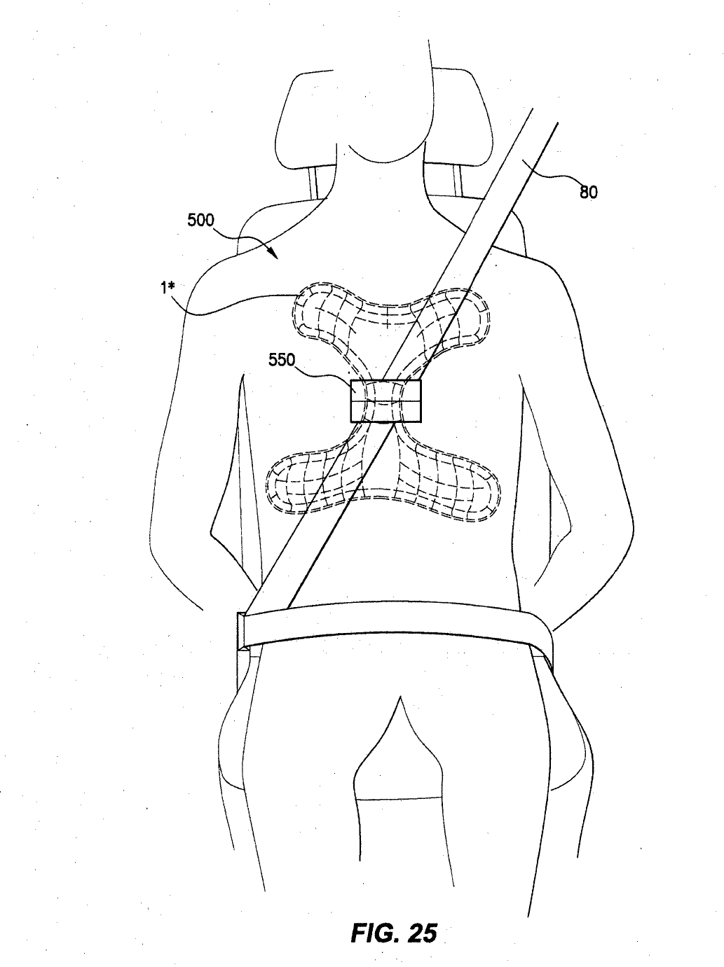

Each of the devices 100, 200, 300 and 400 attaches to the person. However, the invention is also embodied in devices which attach not to the person but to an external object which applies the compressive load to the person's upper front torso. One such device 500, illustrated in FIG. 25, comprises the orthosis 1* and a mounting 550, attached to or integrally formed with the orthosis 1*, that attaches the orthosis 1* to an object, which in the example shown is a seatbelt 80, whereby the orthosis 1* is supported from the object to engage the person's upper front torso adjacent the breasts. In the example shown, the mounting comprises a buckle 550 which is secured to the central portion of the orthosis 1* and is openable to permit the seatbelt strap/sash to be trained therethrough and thence closeable such that the device 500 is coupled to the seatbelt 80 to be supported therefrom. The position of the device along the strap/sash is adjustable by forcing the device 500 sufficiently to overcome the locking effect of the engagement between the strap and mounting. It will be appreciated, again, that the means by which the orthosis 1* is mounted the object may vary considerably without departure from the invention.

In each of the described and illustrated examples which involve a seatbelt, the sash-type seatbelt 80 may be replaced with another form of support or restraint, including but not limited to any one of various alternative types of seatbelt, without departure from the invention. For example, a four-point seatbelt may be employed instead, which seatbelt includes substantially parallel straps which extend downwardly over the person's chest. Advantageously, the orthosis 1*/device is configured such that each of those straps is received over the cephalad and caudal portions on a respective lateral side of the orthosis 1*, whereby again, there is, advantageously, direct load transfer from the seatbelt to the orthosis 1*.

As will be clear, particularly from the examples shown in FIGS. 23 and 25, a panel against which the base of the orthosis 1* is received is not present in all forms of the protective device embodying the invention. In the arrangements shown in those drawings, the orthosis 1* is received directly against the external restraint or support applying the compressive load to the front upper torso.

Preferred embodiments of the invention protect the thoracic cage from injury and trauma under numerous situations, both from, direct trauma from an external source, or from movement within itself. The injury/trauma against which the preferred embodiments protect can comprise Seatbelt syndrome, where both an external loading and internal movement occur, damaging the thoracic cage, laceration and crush of the external tissues, skin, muscles, breast tissue/breast implants, and/or fractures of the sternum or ribs, as well as lung and heart trauma or aorta trauma resulting from the organs ‘slamming’ into the more structurally rigid thoracic cage.

Preferred embodiments of the invention dissipate and redistribute loading through the chest, and potentially minimises the reactive movement of internal organs within it caused generally from inertia forces, greatly diminishing the severity and incidence of many of the injuries seen in such conditions as seatbelt syndrome.

Particular preferred embodiments of the invention provide protection in the context of contact sport, or when loading occurs through the thorax for prolonged periods of high intensity/force, including but not limited to many prone activities such as muscle and joint manipulation in clinical and beauty therapy settings.

Orthoses according to preferred embodiments of the invention are fabricated from a material with excellent compressive and shear resistant qualities, and excellent elastic and recoil properties, allowing dispersal of loading throughout the device, removing focal point loading.

Preferred embodiments of the invention provide excellent products for women with breast augmentation, breast reconstruction, post mastectomy, or any condition where loading of the anterior chest wall causes discomfort, or is potentially damaging to the existing structures of the anterior chest wall, thoracic cage or internal organs.

Particular preferred embodiments of the invention provide protection for individuals with pacemakers, where the seat belt or any form of anterior chest wall may cause discomfort or damage to the device located generally lateral to the sternum are another example of a specific group of individuals who would benefit from its use.

Many modifications will be apparent to those skilled in the art without departing from the scope of the present invention.

1. An orthosis arranged to be received between the upper front torso of a subject and a support through which a load is applied to the upper front torso, to restrict or eliminate stress on the subject's breasts resulting from the load, the orthosis comprising a hollow resiliently deformable body defined by a wall of the orthosis, the body being arranged to contact the subject adjacent the breasts, and resiliently deformable ribbing within the body to support the wall, whereby the orthosis deforms resiliently when so received.

2.-3. (canceled)

4. An orthosis according to claim 1, comprising a substantially open base receivable against the support.

5.-12. (canceled)

13. An orthosis according to claim 1, comprising a central portion extending along a cephalad-caudal axis of the orthosis to be received between the breasts to support the sternum, and:
opposed portions extending bilaterally outwardly from a cephalad end of the central portion to support the clavicle and/or upper ribcage/costal cartilage (“cephalad portions”); and/or
opposed portions extending bilaterally outwardly from a caudal end of the central portion to support the lower ribcage/costal cartilage and/or upper abdomen (“caudal portions”),
each portion comprising a respective part of the body and respective part of the ribbing and being configured such that spaces are defined laterally outward of the cephalad-caudal axis and the breasts occupy the spaces.

14. An orthosis according to claim 13, wherein each said part of the ribbing comprises one or more ribs extending to a base of the orthosis to be compressed between the wall/body and the support.

15. An orthosis according to claim 13, being arranged such that each said portion comprises one or more ribs extending transverse to a respective direction along which that portion engages the subject when the orthosis is so received.

16. (canceled)

17. An orthosis according to claim 15, wherein the or each rib extending transverse is bowed in a direction generally aligned with the respective axis.

18.-21. (canceled)

22. An orthosis according to claim 13, including the caudal portions.

23. An orthosis according to claim 13, including the cephalad portions.

24. (canceled)

25. A method of restricting or eliminating stress on the breasts of a subject resulting from loading on the subject's upper front torso exerted by a support, wherein an orthosis according to claim 1 is arranged between the front upper torso and the support whereby the body contacts the subject adjacent the breasts such that the orthosis deforms resiliently.

26.-31. (canceled)

32. A protective device which comprises an orthosis configured to be received against the upper front torso of a subject adjacent the subject's breasts, so as to restrict or eliminate stress on the breasts resulting from an external load applied to the upper front torso, the device being configured to hold the orthosis removably to the subject or removably to an object applying the load such that the orthosis is so received.

33.-36. (canceled)

37. A device according to claim 32, being receivable around the subject's neck such that the orthosis is hung from the neck.

38. (canceled)

39. A device according to claim 32, wherein comprising strapping or a harness which holds the orthosis and is wearable over the torso.

40. A device according to claim 32, comprising a vest which holds the orthosis.

41.-43. (canceled)

44. A device according to claim 32, wherein said means comprising a mounting for holding the orthosis to a said object.

45. (canceled)

46. A device according to claim 44, wherein configured to hold the orthosis removably to a seatbelt, the seatbelt defining the object.

47.-51. (canceled)
